(19) United States
(12) Patent Application Publication
(10) Pub. No.: US 2015/0157484 A1
(43) Pub. Date: Jun. 11, 2015

(54) SEMI-RIGID SHOULDER BRACES AND RELATED METHODS

(71) Applicant: Clemson University Research Foundation, Clemson, SC (US)

(72) Inventors: Chelsea Lea Ex-Lubeskie, Goose Creek, SC (US); Meredith Cole Donaldson, Westminster, SC (US); Riley Morgan Csernica, Mt. Pleasant, SC (US); Kaitlin Kathleen Grove, Roanoke, VA (US); Charles Alden Thigpen, Simpsonville, SC (US); Joseph J. Calandra, Mt. Pleasant, SC (US)

(21) Appl. No.: 14/626,299

(22) Filed: Feb. 19, 2015

Related U.S. Application Data
(63) Continuation-in-part of application No. PCT/US2013/055777, filed on Aug. 20, 2013.
(60) Provisional application No. 61/691,929, filed on Aug. 22, 2012.
(60) Provisional application No. 61/942,348, filed on Feb. 20, 2014.

(51) Int. Cl. A61F 5/01

(52) U.S. Cl. CPC A61F 5/013

(57) ABSTRACT

Shoulder braces that are configured to allow for a wide range of motion for mobility and stabilization, include a brace body with a torso portion, a plurality of shoulder straps, a substantially rigid shoulder support member releasably held by the brace body. The shoulder support member can have a user-specific custom shape that substantially conforms to external anterior or posterior shoulder anatomy (over the glenohumeral joint) of the shoulder of the user. The brace can also include at least one over the shoulder strap and a torso strap or segment.

### RELATED APPLICATIONS

This application is a continuation-in-part of PCT/US2013/055777, filed Aug. 20, 2013, which claims the benefit of and priority to U.S. Provisional Application Ser. No. 61/691,929, filed Aug. 22, 2012, the contents of which are hereby incorporated by reference as if recited in full herein. This application also claims the benefit of and priority to U.S. Provisional Application Ser. No. 61/942,348, filed Feb. 20, 2014, the contents of which are also hereby incorporated by reference as if recited in full herein.

### FIELD OF THE INVENTION

The present invention relates to shoulder braces.

### BACKGROUND

Each year approximately 71,000 primary anterior shoulder dislocations occur in athletes playing high impact sports and can occur with other at-risk populations as well. See, Lauren Pearson, Young Men and Elderly Women at Biggest Risk for Shoulder Dislocations, Amer. Aca. of Orthopaedic Surgeons, Mar. 1, 2010. These individuals can be at increased risk for recurring dislocations. To lower this risk, patients are often prescribed one of two conventional shoulder orthotics, the SULLY brace or the SAWA brace, both manufactured by DonJoy Orthotics. However, the SULLY device is often thought to be too restrictive and uncomfortable and can impair athletic performance while the SAWA device, while more comfortable, may not provide enough stability to prevent recurrence of a dislocation.

### SUMMARY OF EMBODIMENTS OF THE INVENTION

Embodiments of the present invention provide semi-rigid shoulder braces that provide stabilization of anterior and/or posterior glen-humeral ligaments without immobilizing the shoulder joint.

Shoulder braces for inhibiting secondary anterior shoulder dislocations and/or subluxations (or other shoulder dislocations, e.g., post a secondary injury) are configured to allow for a wide range of motion while also providing shoulder joint stabilization.

Embodiments of the invention are directed to shoulder braces that include a semi-rigid thermoplastic or other shapeable material shoulder support member. The shoulder support member has a user-specific custom shape that substantially conforms to one of a posterior or anterior of a glenohumeral joint of a shoulder of a respective user. The shoulder braces also include a brace body with a plurality of straps that holds the shoulder support member in position over the posterior or anterior of the glenohumeral joint of the shoulder.

The shoulder brace can be for anterior or posterior instability, go on either shoulder, or both shoulders simultaneously

The straps can include at least one over a shoulder strap, and at least one under the shoulder strap.

Some embodiments are directed to shoulder braces with plurality of straps; and a semi-rigid shoulder support member. The semi-rigid shoulder support member can have a user-specific custom shape that substantially conforms to an anterior or posterior of a glenohumeral joint of a shoulder of a respective user. In use, the semi-rigid shoulder support member is sized and configured to reside over the anterior or posterior of the glenohumeral joint of the shoulder of the user.

The straps can include (i) at least one over a shoulder strap and (ii) at least one under the shoulder strap. The over and under the shoulder straps can be back straps.

The over and under the shoulder straps can be attached to the shoulder support member at spaced apart portions of the shoulder support member and to a torso strap or fasteners on a torso portion of a brace body. The over and under the shoulder straps can be configured to extend and retract in a cooperating manner to apply compression against the shoulder support member to hold the shoulder support member against the anterior or posterior of the glenohumeral joint during movement of an arm that is adjacent the shoulder support member.

The straps can include (i) a first elongate strap that is attached to an upper portion of the semi-rigid shoulder support member and (ii) a second elongate strap that is attached to a portion of the semi-rigid shoulder support member below the first strap. The first strap and the second strap can also attached to a torso strap or fasteners on a torso portion of a brace body. The first and second straps can be configured to extend and retract in a cooperating manner to apply compression against the semi-rigid shoulder support member to hold the semi-rigid shoulder support member against the anterior or posterior of the glenohumeral joint during movement of an arm that is adjacent the semi-rigid shoulder support member.

The shoulder support member can have a thickness between about 0.125 inches to about 0.25 inches and has a plurality of vertically spaced apart slots. The slots may include a first upper slot that engages an over the shoulder strap and a second lower slot that engages at least a second strap. The over the shoulder strap can be an over the shoulder back strap for anterior positioning of the shoulder support member. The over the shoulder strap can be an over the shoulder front strap for a posterior positioning of the shoulder support member.

The semi-rigid shoulder support member can be a thermoplastic shoulder support member that is malleable for between 3-5 minutes when heated to between about 65 degrees Celsius to about 70 degrees Celsius for about 1 minute.

The semi-rigid shoulder support member can comprise any suitable material that is moldable to a user-specific shape including air, dry heat, customized injection or other defined molds and the like.

The plurality of straps can include a torso strap, that, in use, defines a closed shape to surround a torso segment of a user at or above a waist of the user, an over a shoulder (back) strap, an under the shoulder (back) strap, an arm band and at least one arm strap. The over and under the shoulder (back) straps and the arm strap can be attached to the shoulder support member. The over and under the shoulder (back) straps can also attached to (a back of) the torso strap while the arm strap is also attached to the arm band and/or the torso strap.

The shoulder brace can include a brace body with at least one sleeve and a torso portion. The sleeve can have a region with a strap attachment segment and the torso portion can have a strap attachment segment.

The plurality of straps can include an over a shoulder strap, an under the shoulder strap, and at least one arm strap. The over and under the shoulder straps and the arm strap can be attached to the semi-rigid shoulder support member. The over and under the shoulder straps can also be attached to the torso attachment segment while the arm strap can also be attached to the arm attachment segment.

The brace body can include left and right side sleeves, each with a strap attachment segment sized and configured to reside proximate a bicep portion of a respective sleeve. The right side strap attachment segment can attach to a right side arm strap for a right side anterior or posterior shoulder brace configuration. The left side strap attachment segment can attach to a left side arm strap for a left side anterior or posterior shoulder brace configuration.

The brace body can have a shirt configuration that provides the torso portion and right and left arm sleeves. The brace body can include right and left side pockets to releasably engage a respective semi-rigid shoulder member.

The at least one semi-rigid shoulder support member can be attached to three strap segments including an over the shoulder strap segment, an under the shoulder strap segment and an arm strap segment.

The under the shoulder strap segment and the arm strap segment can be attached to a lower end portion of the semi-rigid shoulder support member. The over the shoulder strap segment can be attached to an upper end portion of the semi-rigid shoulder support member.

The under and over the shoulder strap segments and the arm strap segment can be fixedly attached to the semi-rigid shoulder support member.

The shoulder brace can have a torso strap. The torso strap can include diagonally oriented, parallel loop segments that attach to the over and under the shoulder back strap segments.

Some embodiments are directed to shoulder braces that include a shoulder support member; a torso strap or torso portion of a brace body with a strap attachment feature; an elongate over a shoulder strap comprising opposing longitudinally spaced apart first and second end portions, the first end portion connected to the torso strap or torso portion and the second end portion connected to the shoulder support member; and an arm strap that is connected the shoulder support member and an arm band or sleeve of a brace body.

The shoulder brace can also include an under the shoulder strap comprising opposing longitudinally spaced apart first and second portions, the first portion can be connected to the torso strap or torso portion of the brace body and the second portion can be connected to the shoulder support member at a location that is below the second end portion of the over the shoulder strap.

The shoulder support member comprises an upper laterally extending slot that engages the over the shoulder trap and a lower curvilinear slot that engages the arm strap and the under the shoulder strap.

The shoulder brace can have a brace body that has a shirt configuration with at least one arm portion with a sleeve and a torso portion. The at least one arm portion can have a strap attachment feature for the arm strap. The torso portion can have the torso strap attachment feature for the first end portions of the over and under the shoulder straps.

The brace body with the shirt configuration can have right and left arm sleeves. The brace body can include right and left side pockets to releasably engage a respective semi-rigid shoulder member.

The under and over the shoulder straps and the arm strap are fixedly attached to the shoulder support member.

The shirt configuration can allow for anterior and posterior arrangements of the shoulder support member and/or bilateral arrangements of respective straps and shoulder support members.

The shoulder brace can be used for anterior or posterior positioning of the shoulder support member.

Other embodiments are directed to methods of providing support to a shoulder of a user. The methods include: (a) providing a shoulder brace with a semi-rigid thermoplastic shoulder support member that resides over an anterior or posterior glenohumeral joint of a user with a compromised shoulder; (b) allowing a functional, substantially full range of motion when the user wears the shoulder brace; and (c) providing compressive support to a glenohumeral joint of the compromised shoulder.

The providing the shoulder brace can be carried out by providing a plurality of cooperating straps including an arm strap, an over the shoulder strap and an under the shoulder strap that are attached or attachable to the shoulder support member. The method can include: attaching arm strap to a sleeve of a brace body or to an arm band on an arm of a user adjacent a compromised and/or injured shoulder; and attaching the over and under the shoulder traps to a torso strap or a torso portion of a brace body. The providing compressive support can be carried out by allowing the under and over the shoulder strap to change in length, one lengthening while the other shortening to retain the shoulder support member over the target joint during abduction.

The over the shoulder strap can be attached to an upper portion of the shoulder support member and the under the shoulder strap and the arm strap can be attached to a portion of the shoulder support member, under the over the shoulder strap.

The method can include attaching a motion-limiting restraint strap to the arm band or to the sleeve and a front segment of the torso strap or the torso portion of the brace body.

Still other embodiments are directed to methods of providing a customizable shoulder brace for treating a subject at risk for shoulder dislocation or needing support of a shoulder. The methods can include: providing a shoulder brace; shaping a shoulder support member to conform to a shape of external anatomy over a glenohumeral joint of a shoulder of a patient; and placing the shaped shoulder support member anterior or posterior to a user's shoulder over the glenohumeral joint and holding the shoulder support member in position using the shoulder brace.

The providing can provide at least one moldable shoulder support member as the thermoplastic shoulder support member in a kit to allow for custom molding of a respective shoulder support member for a respective user. The thermoplastic shoulder support member has a thickness between about 0.125 inches to about 0.25 inches. The thermoplastic shoulder support member is malleable for between 3-5 minutes when heated to between about 65 degrees Celsius to about 70 degrees Celsius for about 1 minute.

The at least one moldable shoulder support member can be provided in the kit pre-attached to three strap segments including an over the shoulder strap segment, an under the shoulder strap segment and an arm strap segment

The under the shoulder back strap segment and the arm strap segment can be fixedly attached to a lower end portion of the shoulder support member and the over the shoulder back strap segment can be fixedly attached to an upper end portion of the shoulder support member.

The shoulder brace can include a brace body having a shirt configuration with at least one sleeve and at least one pocket, the pocket can releasably engage the shoulder support member.

The shoulder brace can include only a plurality of cooperating straps to hold the shoulder support member in position.

The shoulder support member can include a plurality of laterally extending slots that engage straps of the shoulder brace.

The at least one moldable shoulder support member can be provided in the kit pre-attached to three strap segments including an over the shoulder strap segment, an under the shoulder strap segment and an arm strap segment.

Some embodiments are directed to shoulder braces that include a brace body with an arm portion configured to reside about at least one shoulder of a user and a torso portion. The braces include a plurality of shoulder straps extending over a respective arm portion of a shoulder and a substantially rigid thermoplastic shoulder support member releasably held by the arm portion of the brace body. The shoulder support member has a user-specific custom shape that substantially conforms to a shoulder of the user. The brace also includes at least one adjustable length laterally extending torso strap attached to the torso portion.

The brace body can include an expandable, breathable compressive fabric.

The at least one torso strap can be a single strap that has an adjustable length and resides on only one side portion of the brace body. An opposing side portion can include two long edge portions that are releasably attachable.

The plurality of straps can include a plurality of closely spaced straps (typically between 3-7) that extend from a front upper portion of the brace body to a rear upper portion of the brace body and that are sized and configured to provide stabilization of ligaments. The straps have adjustable lengths.

The shoulder support member can substantially conform to an underlying glenohumeral joint of the user/patient.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems and/or methods according to embodiments of the invention will be or become apparent to one with skill in the art upon review of the following drawings and detailed description. It is intended that all such additional systems, methods, and/or devices be included within this description, be within the scope of the present invention, and be protected by the accompanying claims.

### BRIEF DESCRIPTION OF THE DRAWINGS

Other features of the present invention will be more readily understood from the following detailed description of exemplary embodiments thereof when read in conjunction with the accompanying drawings.

FIG. 1A is a front view of a right shoulder brace according to embodiments of the present invention.

FIG. 1B is an enlarged view of the brace shown in FIG. 1A illustrating a location of an (internal) shoulder support member according to embodiments of the present invention.

FIG. 2 is a front view of the device shown in FIG. 1A, shown on a patient/user according to embodiments of the present invention.

FIG. 3 is a top view of an exemplary shoulder support member pocket on a shoulder portion of the device shown in FIG. 1A according to embodiments of the present invention.

FIG. 4A is a side view of a shoulder brace member before being formed with a custom user shoulder shape.

FIG. 4B is a side view of the shoulder brace member shown in FIG. 4A after formed to have a custom shape that confirms to a user's shoulder according to embodiments of the present invention.

FIG. 5A is an anterior perspective view of the brace shown in FIG. 1A.

FIG. 5B is a posterior perspective view of the brace shown in FIG. 1A.

FIG. 6A is a graph of shoulder range of motion (ROM) in degrees (average) by motion exercise type allowed by no brace versus the semi-rigid shoulder brace shown in FIG. 1A (n=7) according to embodiments of the present invention.

FIG. 6B is a table that shows average range of motion for subjects (n=7) using the brace shown in FIG. 1A according to embodiments of the present invention.

FIG. 7 is a graph of time (average) for a user to self-attach the high-impact, semi-rigid brace for five different trials according to embodiments of the present invention.

FIG. 8 is a flow chart of exemplary operations that can be used to carry out embodiments of the present invention.

FIG. 9A is a front perspective view of a clinician or other person molding the shoulder brace member to a user's anterior glenohumeral joint shape according to embodiments of the present invention.

FIG. 9B is a top perspective view of the shoulder brace member shown in FIG. 9A in a custom form shape ready for attachment to the brace according to embodiments of the present invention.

FIGS. 10A and 10B are sequential front perspective views of a user self-applying (donning) the brace according to some embodiments of the present invention.

FIG. 11 is a front view of another example of a shoulder brace member adapted to sit over the glenohumeral joint according to embodiments of the present invention.

FIG. 12 is a front view of an exemplary shoulder brace with a brace body comprising a series of cooperating straps according to embodiments of the present invention.

FIG. 13 is an exploded, front perspective view of an exemplary shoulder brace shown in FIG. 12.

FIG. 14 is a rear view of a shoulder brace with a brace body of cooperating straps shown on a user according to embodiments of the present invention.

FIG. 15 is a front view of the shoulder brace shown in FIG. 14 also shown on a user according to embodiments of the present invention.

FIG. 16 is a front view of the shoulder brace shown on the user in FIG. 15 with an additional and optional restraint for limiting range of motion according to some embodiments of the present invention.

FIGS. 17A, 17B, and 18-20 are front views of exemplary cooperating straps, some or all of which can directly or indirectly be attached together for a shoulder brace according to embodiments of the present invention.

FIG. 21 is a front view of an assembled shoulder brace as worn by a user according to embodiments of the present invention.

FIG. 22 is a rear view of the assembled brace shown in FIG. 21 as worn by a user according to embodiments of the present invention.

FIG. 23 is a schematic illustration of a portion of the shoulder brace with affixed strap portions and with a malleable shape (e.g., moldable thermoplastic member) shoulder support member as a strap sub-assembly according to embodiments of the present invention.

FIG. 24 is a schematic illustration showing that the shoulder support member can be removed from the heat source such as that shown in FIG. 23, then placed over the glenohumeral joint for custom shaping the malleable/moldable member to a user's joint shape according to embodiments of the present invention.

FIG. 25 is a rear view of an assembled shoulder brace similar to that shown in FIG. 21, but illustrating it can be configured for bilateral use according to embodiments of the present invention.

FIG. 26 is a rear view of an assembled shoulder brace similar to that shown in FIG. 21, but illustrating it can be configured for posterior use according to embodiments of the present invention.

FIG. 27A is a front view of a shoulder brace for anterior according to some embodiments of the present invention.

FIG. 27B is a back view of the shoulder brace shown in FIG. 27A according to embodiments of the present invention.

FIG. 28A is a front view of a shoulder brace for posterior support according to some embodiments of the present invention.

FIG. 28B is a back view of the shoulder brace shown in FIG. 28A according to embodiments of the present invention.

FIG. 29A is a front view of a bilateral shoulder brace according to some embodiments of the present invention.

FIG. 29B is a back view of the shoulder brace shown in FIG. 29A according to embodiments of the present invention.

### DETAILED DESCRIPTION OF EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. One or more features shown and discussed with respect to one embodiment may be included in another embodiment even if not explicitly described or shown with another embodiment.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “about” means that the recited number or value can vary by +/−20%.

Embodiments of the invention are particularly suitable for human uses and/or some veterinary uses.

The shoulder permits a very large range of motion. The range of motion permitted by the shoulder provides substantial utility of the arms of humans. Arms are used in many activities in daily living as well as for sports for throwing, or batting objects such as balls, or for propelling the body in sports such as swimming.

Performing at a high level in many activities requires a full range of motion of the shoulder. The shoulder is subject to injuries that limit the range of motion. The large range of motion of the shoulder also subjects the shoulder to physiological damage due to mechanical stresses placed on joints and ligaments that make up the shoulder. These stresses are at their highest during physical activities in which a participant is attempting to perform at his or her highest levels. In particular, the glenohumeral joint is subject to instability. The glenoid fossa forms a very shallow socket, requiring it to be held together by extensive ligament and muscle attachments. Certain types of force, such as sudden jerks by high forces, can easily weaken or damage the shoulder. In cases where the ligaments, cartilage or muscles that support the glenohumeral joint are damaged or weakened, external support is useful.

Embodiments of the invention provide a functional shoulder brace that provides compressive support to a damaged or weakened glenohumeral joint, while allowing a substantial range of motion that makes the brace useful for engaging in physical activity.

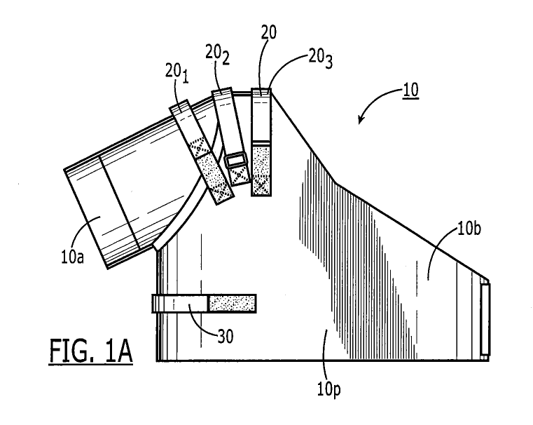

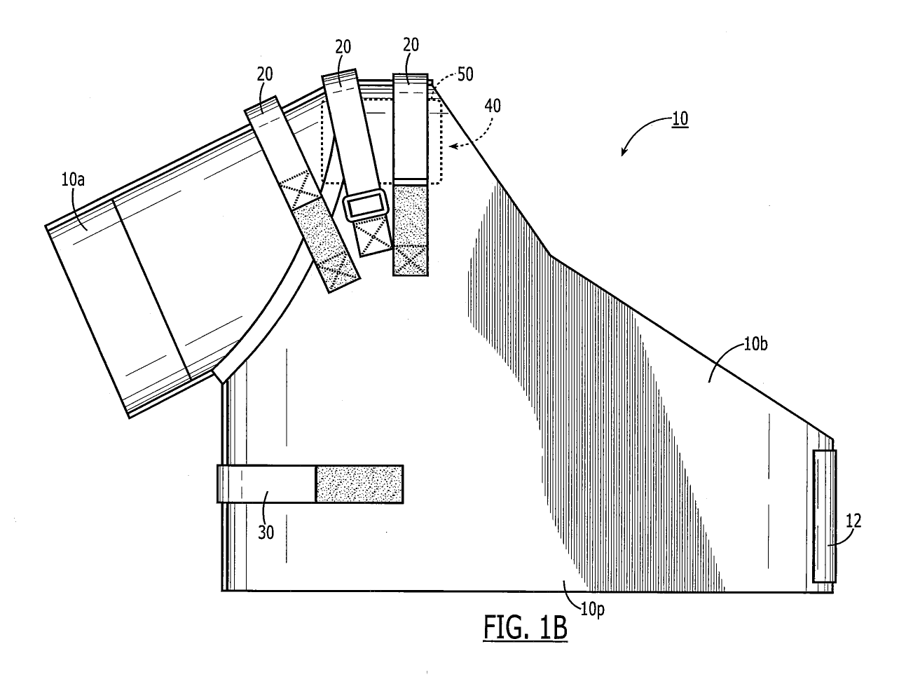

Turning now to the figures, FIGS. 1A and 1B illustrate an example of a shoulder brace 10 according to embodiments of the present invention. The brace 10 can be light-weight such as under about 1 pound, including, for example, about 0.75 pounds, about 0.5 pounds (about 170 grams), about 0.25 pounds or even less. The brace 10 can include a brace body 10b that holds an shoulder support member 40 over the anterior glenohumeral joint. The shoulder support member 40 is typically a semi-rigid or rigid shape-moldable member such as a thermoplastic shoulder support member.

The brace body 10b can comprise a flexible (stretchable) compressive material, typically a material that is “breathable” for user comfort. The brace body 10b can include a single arm portion 10a that fits onto a user's arm and a torso portion 10p. In some embodiments, the torso portion 10p can include at least one longitudinally extending side 11 with longitudinally extending edge portions 12 that are releasably attachable together to allow a user to relatively easily take the brace on and off. VELCRO® or hook and loop attachments can be used. In other embodiments, buckles, buttons, zippers or snaps (alone or in any combination) can be used. The torso portion 10p may include only a strap 30 or an attachment section 130 that can be incorporated into a shirt or vest-like garment to partially or totally surround a torso or trunk of a body, e.g., a waist and/or chest f a human user (e.g., define a closed loop) at or above the waist, typically it can reside mid-chest, above the waist and over or below the sternum of a user (FIGS. 1, 14, 15, 21, 22, 27A, for example).

In some embodiments, the brace body 10b can include breathable fabrics such as neoprene or other materials that have “stretch” or elastomeric fibers such as spandex that may provide a compression-type or snug fit. LYCRA® is an example of a spandex stretch fiber. Other stretch fibers may also be suitable. Such stretch fibers may include, but are not limited to, fibers presently classified in the polyester textile label classification of the U.S. Federal Trade Commission, but under which a new subclass of “elasterell-p” has been proposed; these, may also be suitable as stretch fibers as the are described as inherently elastic, bicomponent textile fibers consisting of two substantially different forms of polyester fibers (DuPont's version of this fiber is referred to as “T400”).

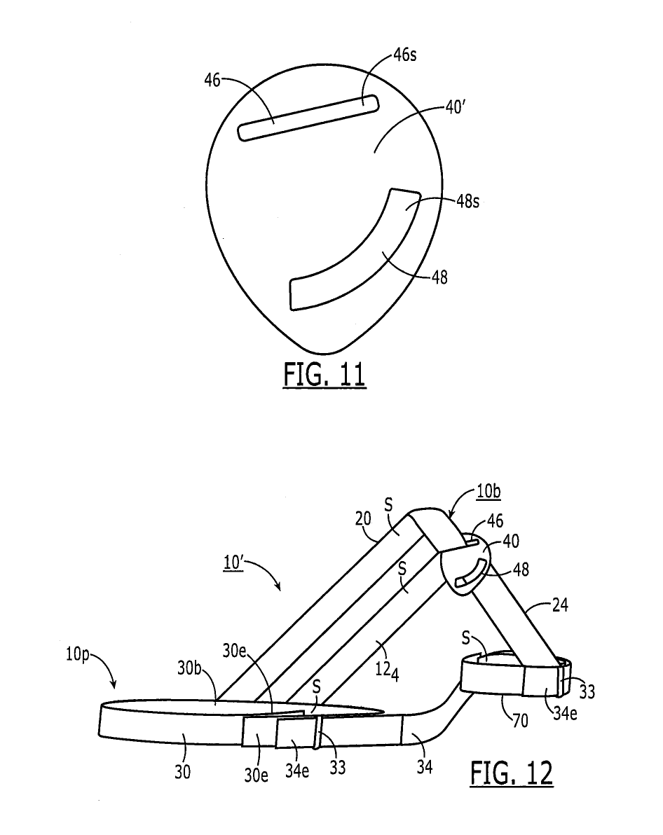

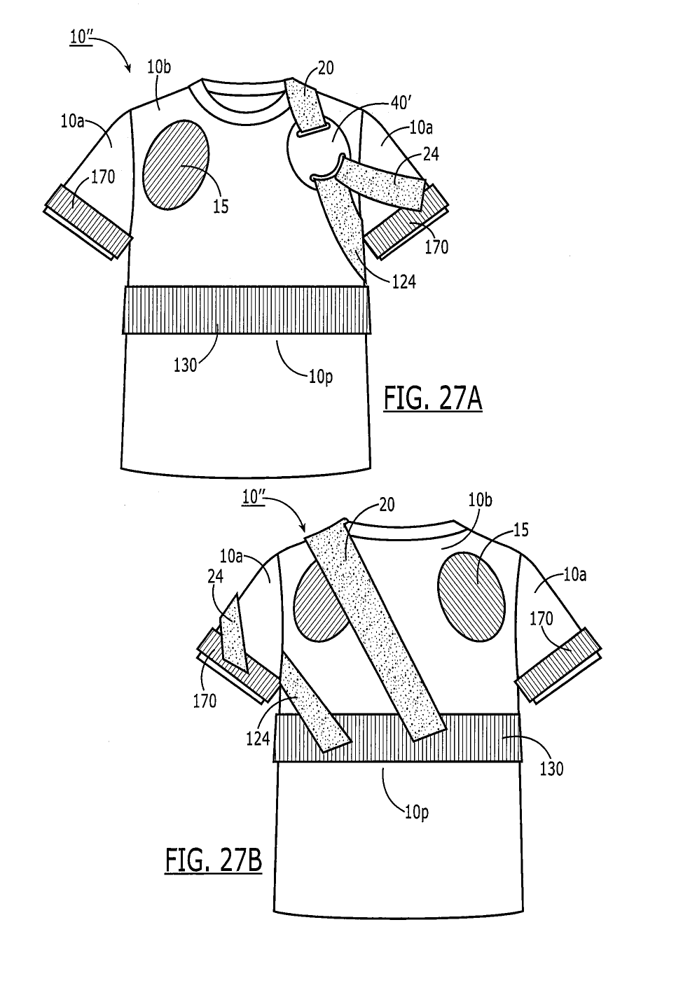

In some embodiments, the brace body 10b can include a zipper that attaches the two edge portions 12. The two edge portions 12 may be on the side or may reside in a center or other user accessible portion of the torso portion 10p brace body to allow for easy donning and removal. In other embodiments, as will be discussed below, the brace body 10b can comprise cooperating straps 20, 30 and the like, without requiring a vest-like body (e.g., FIG. 12) or can include a torso attachment section 130 that may be held on a supporting substrate such as a wearable garment like a shirt or vest (e.g., FIG. 27A).

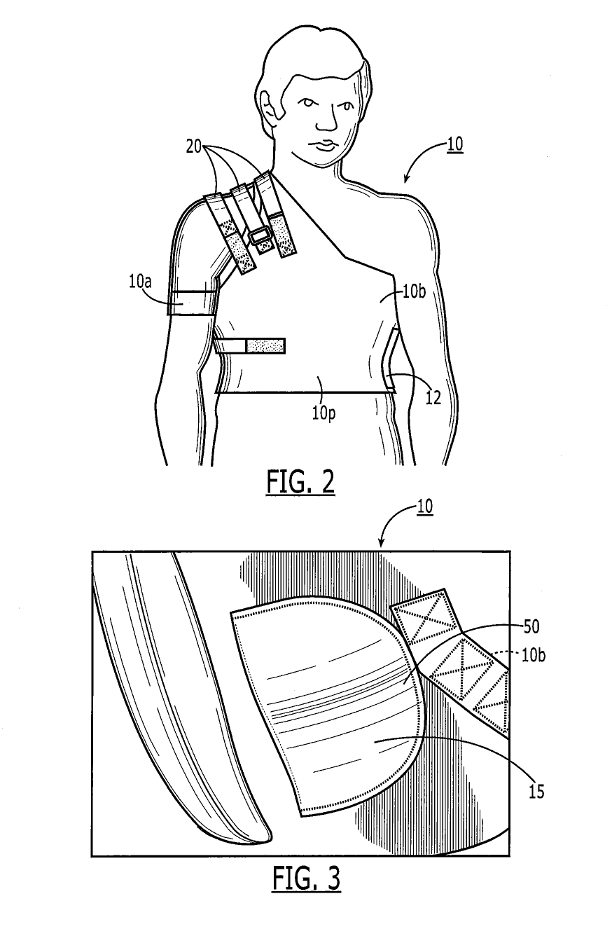

The brace body 10b can include portions that are compressive and portions that are not. The brace body 10b can be a monolithic single layer or multi-layer configuration. The brace body 10b can include a plurality of different materials, defining different portions of the brace body. In some embodiments, the brace body 10b includes a pocket 15 for a shoulder support member 40 (FIGS. 3, 4).

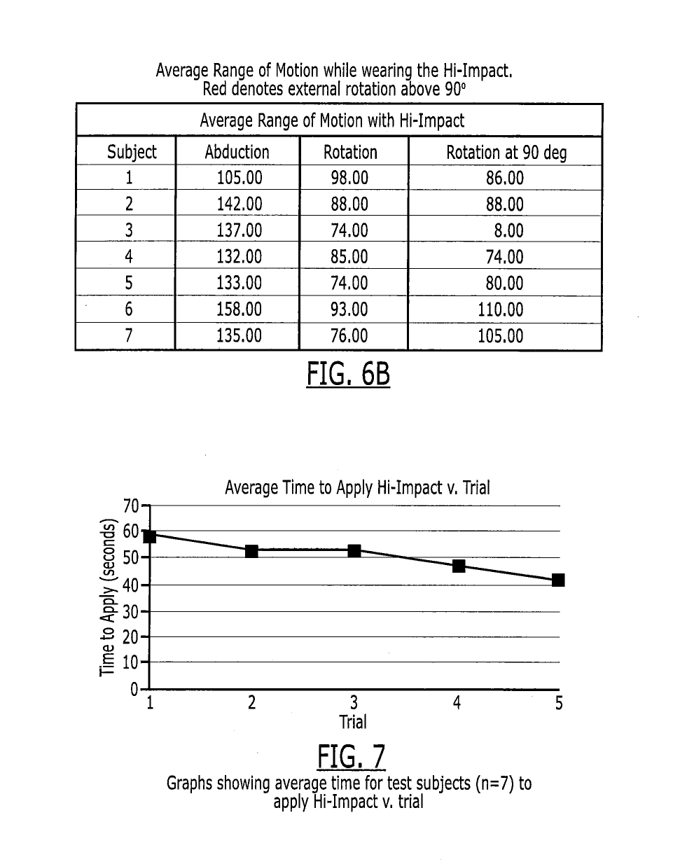

As shown in FIGS. 1A, 1B and 2 the brace 10 can include at least one, shown as a plurality, length-adjustable shoulder strap 20 that extends over the shoulder. The straps 20 can allow a user or clinician to control the tightness of the brace 10 to provide adequate shoulder support while allowing an average of about 110 degrees abduction and about 90 degrees external rotation (see FIG. 6).

The brace 10 can be configured to control a range of motion so that simultaneous abduction of greater than 90 degrees and external rotation of greater than 90 degrees is inhibited. The at least one shoulder strap 20 can include at least two straps (shown as three straps 20₁, 20₂, 20₃) although more than three straps 20 may be used. The shoulder straps 20 can be oriented across the shoulder to mimic the natural function of glenohumeral ligaments. The plurality of shoulder straps can include between 3-7 shoulder straps.

It is contemplated that the brace can provide up to about 770 N of (dynamic) support, typically between about 600 N to about 770 N of support, although other support ranges may be provided.

The brace 10 can optionally include at least one laterally extending adjustable torso strap 30 that resides proximate to but under the arm at an upper to medial portion of the torso. The at least one torso strap 30 can be a single strap or more than one strap. Where multiple torso straps 30 are used, they can reside on a single side of the brace body, typically on the same side as the shoulder straps 20 or on different sides (or even front or back). The torso strap 30 can be used to tighten the brace to conform to the user while allowing for resilient chest expansion during breathing. The torso portion 10p of the brace body 10b with the torso strap 30 can allow for outward expansion of up to about three inches. In other embodiments, the torso portion of the brace may not require or use torso straps.

The brace body 10b can optionally include a pocket 15 that can be an externally accessible pocket that may optionally be formed in compressible material. The compressible material may be that of the brace body 10b. However, as shown in FIG. 3, the pocket 15 can be formed using an inner layer of material 50 (such as nylon) that is attached to an anterior (internal surface or side) of the arm portion 10a of the brace body 10. The term “pocket” is used broadly and refers to any structure or compartment that (detachably and/or releasably) holds the shoulder support member 40, 40′ and is not required to have a continuous outer surface. The pocket 15 can be sewn or chemically (e.g., adhesively) attached to a strap or a primary body of the brace or formed in other manners as will be known by those of skill in the art. The pocket 15 can be an interior nylon pocket configured with one open portion that can be releasably attached or sized to retain the shoulder support member but also allow the shoulder support member 40 to be removed for washing the brace body 10b and/or to replace a respective member 40 and/or form an unformed member to substantially conform to the patient's anatomical shoulder shape.

In other embodiments, the shoulder support member 40, 40′ can comprise integral mounting components such as a surface with VELCRO® type fasteners, apertures, fabric hooks or loops that attach to an underlying or overlying surface of the brace body and/or strap(s) with cooperating mounting features, e.g., straps ends, loops or hooks, adhesives or other mounting configurations that do not require a pocket.

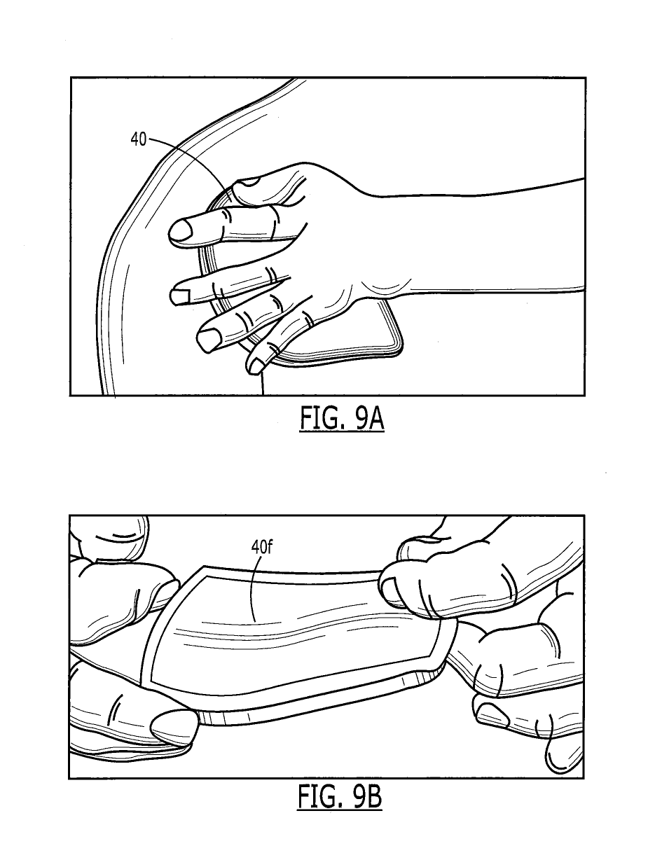

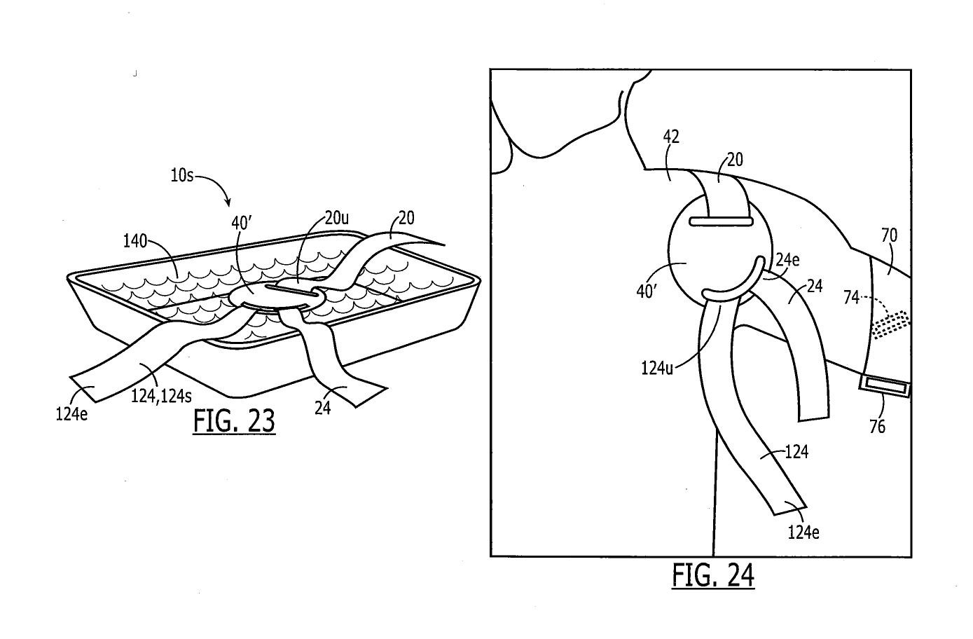

As shown in FIGS. 4A and 23, for example, the shoulder support member 40, 40′ can be provided as a solid (or hollow), semi-rigid or rigid device with a first shape (typically substantially planar) prior to use in a kit or package that can be custom shaped to a user's shoulder prior to use of the brace 10. FIG. 4B illustrates the shoulder support member having a post-molded shape 40f. FIG. 9A illustrates the shoulder support member 40 being molded to conform to a patient's shape at forward portion of the shoulder (below the top) at/over the glenohumeral joint. In some embodiments, the shoulder support member 40 resides over the (anterior or posterior) glenohumeral joint and does not extend over the top of the shoulder or down the arm of a user.

The shoulder support member 40 can be semi-rigid. The shoulder support member can be sized and configured to substantially conform to external anterior shoulder anatomy over the glenohumeral joint. The term “semi-rigid” when referring to the shoulder support member 40, 40′ means that the shoulder support member body may flex when sufficient force is applied thereto but has a three-dimensional body with a fixed shape and sufficient rigidity to retain the fixed three-dimensional body shape (and is typically a self-supported fixed shape apart from the brace) when exposed to forces from normal physical activity.

In some embodiments, when heated to a defined temperature (above room temperature), the member 40, 40′ can be sufficiently malleable or pliable to be able to change in shape when a sufficient (typically manual) compressive force is applied. The word “malleable” means that at least a portion of the member 40, 40′ is able to change in shape, e.g., is moldable, so as to be able to be pressed permanently out the original shape to have a custom shape conformable to the posterior or anterior glenohumeral joint of a user without breaking and/or cracking.

FIG. 9B illustrates the post-mold shape 40f ready for use with the brace 10. In FIG. 9B, the shoulder support member 40 is shown upside down and in use it is rotated about 90 degrees upward with the cavity facing the shoulder of the user.

The shoulder support member 40 can be a thermoplastic shoulder support member of suitable size and shape, e.g., between about 0.125 inches to about 0.50 inches thick, typically between about 0.125 to about 0.25 inches thick. The shoulder support member 40, 40′ can be a low-temperature thermoplastic material of synthetic or natural material, such as polymers and/or rubbers. In some embodiments the thermoplastic material can comprise MARQUE EASY™ thermoplastic material from Chesapeake Medical Products, Inc. In use, the shoulder support member 40 can be substantially rigid while the straps and/or underlying or other part of the brace body 10b can be flexible allowing for mobility of the joint with stabilization.

Although not required, the shoulder support member 40, 40′ may optionally include a cushion layer or attachment, such as a solid gel or neoprene material that provides padding for additional comfort.

In some embodiments, the shoulder support member 40, 40° can be heated to between about 65° C.-70° C. (150° F.-160° F.) for a about 1 minute to become malleable and/or moldable and allows for a work time of between about 3-5 min to form the desired shape.

The area of the shoulder support member 40, 40′ may vary depending on size of the brace (e.g., XS, S, M, L, XL and the like) but typically has an area that is between about 10 in² to about 20 in². In particular embodiments, the shoulder support member 40, 40′ can be about 0.125 inch thick with about a 3×5 inch perimeter and may have a substantially oval shape. In other embodiments, other geometric shapes such as polygons can be used. In some embodiments, the shoulder support member 40, 40′ can be a custom-fitted shoulder support member that substantially conforms to a user's shoulder (external anatomy over a humeral head or over the glenohumeral joint) to increase support and proprioception. In some embodiments, the shoulder support member 40, 40′ can be removed from the brace, e.g., from its designated pocket 15 or detached from strap(s). In other embodiments, the shoulder support member 40, 40′ can be affixed and non-removable from a portion of the body of the brace.

The shoulder support member 40, 40′ cooperates with the brace body 10b providing a semi-rigid device that provides support for weakened glenohumeral ligaments and/or anterior or posterior shoulder ligaments as a result of dislocations, for example.

While particularly suitable for athletes experiencing or at-risk for recurring (particularly, anterior) shoulder dislocations, the braces 10 are also suitable for anyone desiring additional shoulder support.

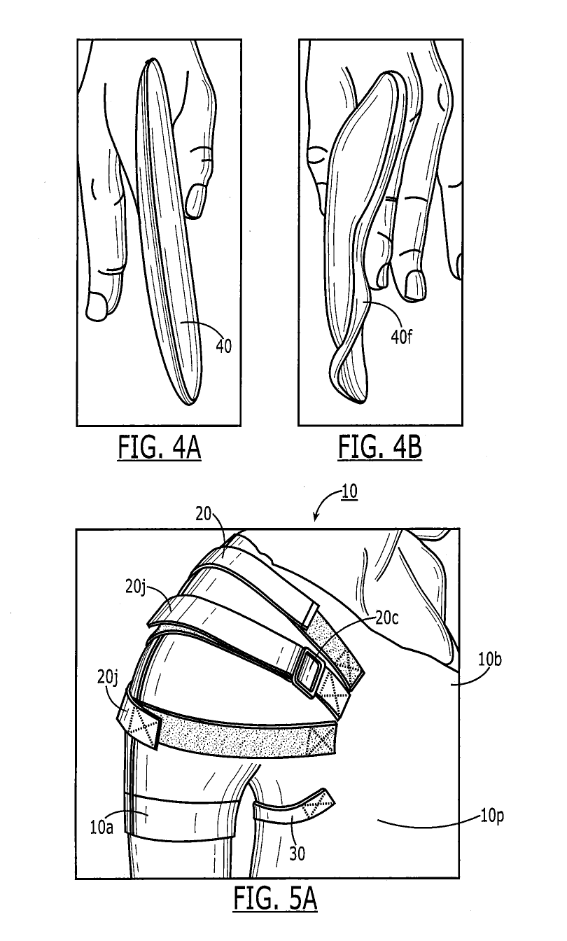

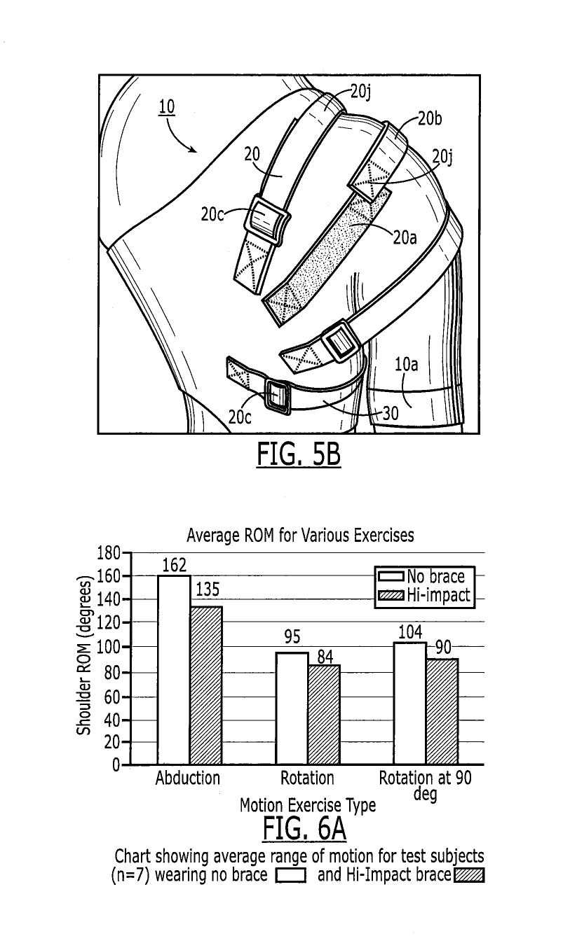

As shown in FIGS. 5A and 5B, the (over the) shoulder strap(s) 20 and torso strap 30 can comprise nylon. The shoulder strap 20 and may include one side that has adjustable length with corresponding pairs of strap legs 20a, 20b attachable at a joint 20j by hook and loop type attachment. The straps 20 can be configured so that a buckle 20c (allowing for adjustable length of one side of the strap) is on the first end 20a on one strap and on the second end 20b on the adjacent strap. In some embodiments, other releasable attachment members can be used in lieu of or with the hooks and loops and other adjustable length members can be used in lieu of buckles. The straps 20 can apply pressure against the underlying shoulder support member 40 to keep the shoulder support member in position and/or apply stabilizing forces against the humeral head of the shoulder.

The brace 10 can restrict abduction and external rotation of the shoulder joint to about 90 degrees combined abduction and external rotation, which can provide stabilization in the area of the anterior glenohumeral ligaments and provide sufficient force to keep the shoulder in place without immobilizing the joint.

In some embodiments, the brace body 10b can comprise a breathable neoprene fabric which is lightweight and has a low profile fit allowing it to fit under athletic clothing and/or padding such as those often worn by athletes.

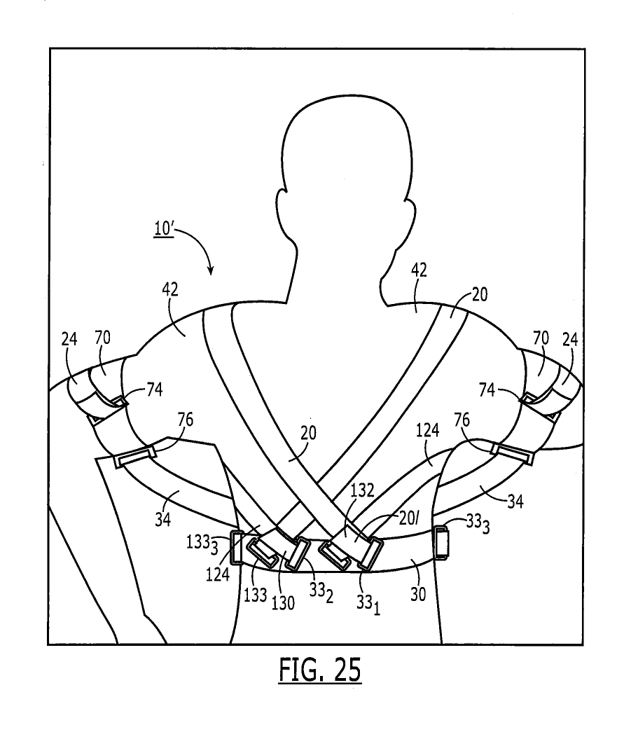

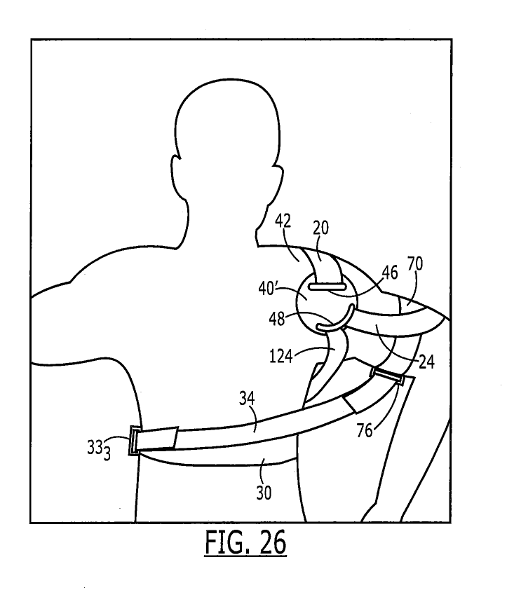

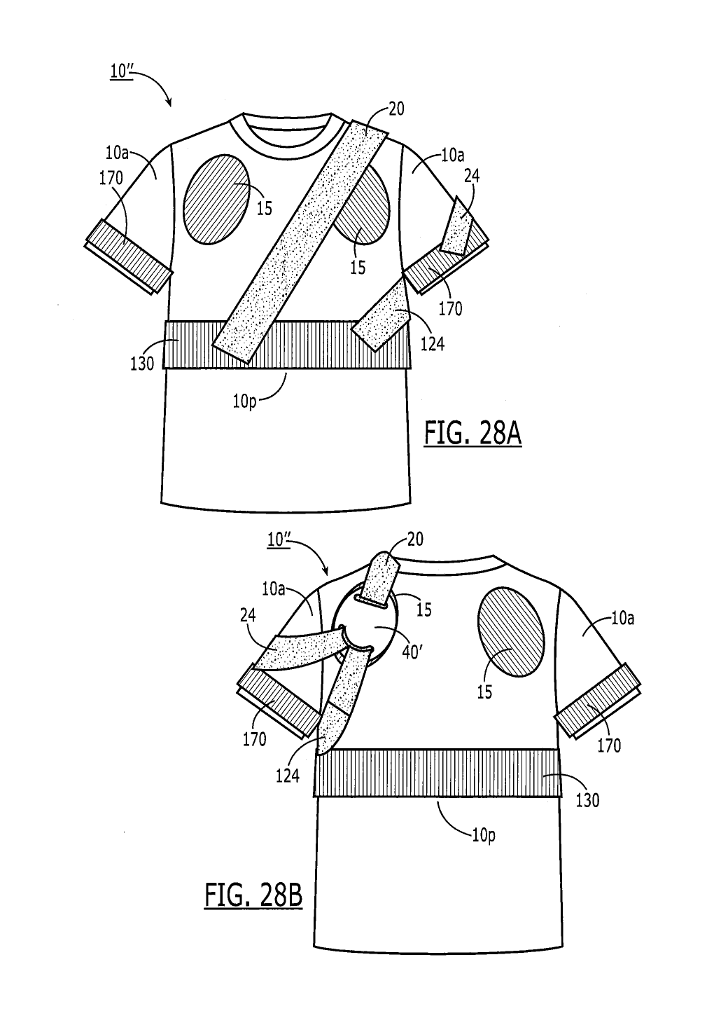

FIGS. 1A, 1B, 2, 5A and 5B show a right shoulder brace 10. The left shoulder brace is similarly configured but with the arm 10a on the left side of the brace body 10b. The shoulder brace can also be configured in the reverse to hold the shoulder member 40, 40′ over a posterior glenohumeral joint (FIG. 26, FIGS. 27A, 27B) or hold bilateral pairs of the shoulder support members 40, 40′ concurrently (e.g., FIGS. 25, 28A, 28B).

The brace 10 can be provided in a range of sizes to accommodate different size users, e.g., extra small (XS), small, medium, large, extra large and even larger (typically 2XL and 3XL). The brace 10 can be provided in pediatric and adult size ranges and/or female to male configurations. Female configurations may have a different torso shape or size. Female adult sizes may be provided in a range of cup and chest sizes for better form fit where “vest” type brace bodies are used.

FIG. 6A is graph that shows the relative mobility allowed by the brace 10 through a wide range of motion for different exercise types allowing for a user to participate in most sport activities, substantially comparable to people not wearing a brace, when wearing the braces 10.

FIG. 6B is a table of average range of motion of different users wearing the brace. The braces 10 can reduce the average range of motion for all subjects in all natural movements. Each subject completed a series of range of motion tests while wearing the brace 10 shown in FIGS. 1A/1B with the custom formed shoulder support member 40. Comparison tests were then performed to find each subject's baseline. The range of motion for adduction was tested using a goniometer to measure deflection of the arm from 0 degrees at rest by the side to the maximum in-plane deflection. The amount of external rotation was measured with the humerus hanging at the side and the elbow bent 90 degrees, parallel to the ground. The amount of external rotation was measured with the humerus held at 90 degrees adduction, and the forearm at 90 degrees to the humerus. The motions were repeated three times and the average deflection was calculated. The brace 10 can reduce the average range of motion for most users to 90 degrees showing the ability of the brace to support and improve stability during these motions in the glenohumeral joint.

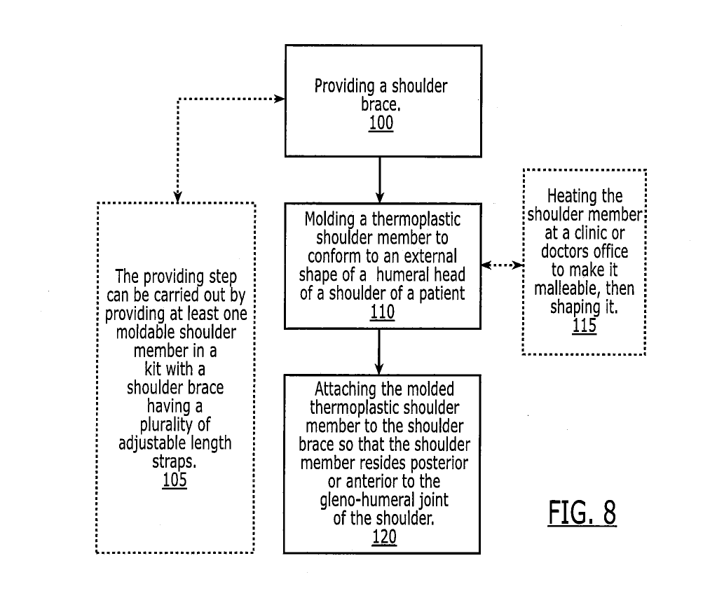

In some embodiments, the brace 10 is configured to allow for relatively quick donning and removal (e.g., on and off) times by a person without requiring any assistance for most users. This convenient self-assembly and removal can promote compliance and potentially avoid recurrence of shoulder dislocations. FIG. 7 illustrates that users can easily self-attach the brace 10 in about 1 minute or less.

FIG. 8 illustrates exemplary steps or operations that can be used to treat a subject (e.g., an athlete) at risk for shoulder dislocation or needing support of a shoulder. A shoulder brace with a torso portion is provided (block 100). A thermoplastic (or other suitable material) shoulder support member can be molded to conform to a shape of (external anatomy over) a glenohumeral joint of a shoulder of a patient (block 110). The molded (thermoplastic) shoulder support member can be held by the brace 10 so that the shoulder support member resides posterior and/or anterior to the shoulder over a glenohumeral joint (block 120).

The providing step can be carried out by providing at least one moldable shoulder support member in a kit with a brace body which can be a series or cooperating straps and/or a compressible breathable fabric shoulder brace body. The shoulder brace can include a single one or a plurality of adjustable length shoulder straps that position the shoulder support member 40, 40′ in the appropriate location and the method can include adjusting the strap(s) to provide desired support and compressive strength of the shoulder support member against the shoulder (block 105).

The shoulder support member can be heated at home and/or a clinic or doctor's office to make it malleable, then the shoulder support member can be shaped for the molding step (block 115). The brace can be provided in a package with instructional media such as, for example, a training manual, template or instruction sheet or brochure and/or a web link to a video that shows how to mold the shoulder support member and/or how to don the brace (e.g., a preferred order of steps for a user to take to mold the shoulder support member and/or to put on the brace). The shoulder support member may also come with indicia of orientation for placement into the brace (e.g., pocket) such as “up” or “down” or “faces in” or “faces out” surface markings (arrows, icons or images may also be used). The straps or portions thereof may also be color-coded so that a user knows which ends to attach together, for example.

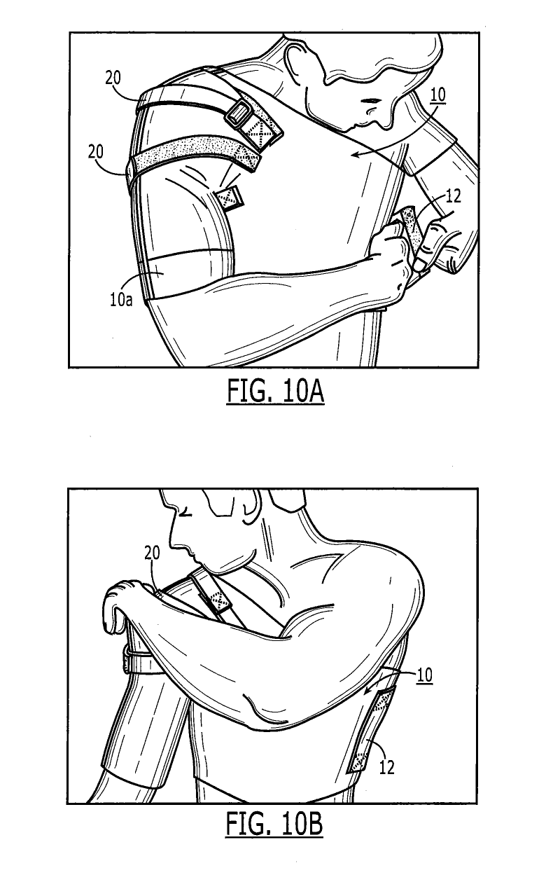

FIGS. 10A and 10B illustrate an example of a patient/user self-applying (donning) some embodiments of the brace 10. In some embodiments, a user slips an arm through the sleeve (with the straps 20 loose), pulls the body 10b closed on the opposing side and attaches the side 12. The user then tightens the straps 20 and strap 30 (where used) as needed.

FIG. 11 illustrates another embodiment of the shoulder brace 10′ with a support member 40′ that can be used with a brace body 10b to provide compressive support of a glenohumeral joint. The shoulder support member 40′ can include two attachment segments 46, 48 that attach to different defined straps.

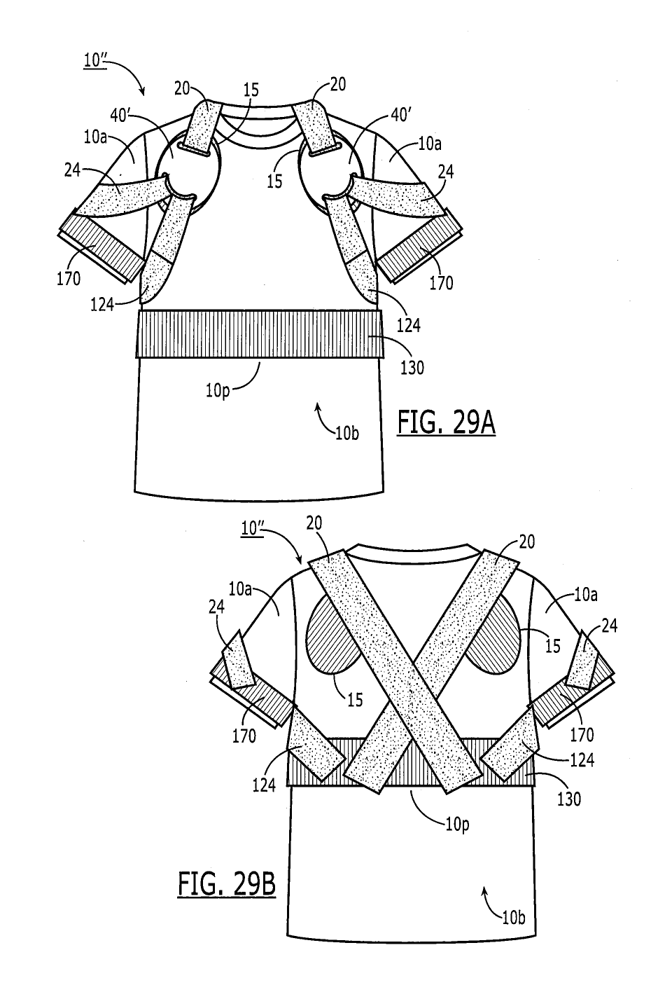

FIGS. 12-21 illustrate that the brace body 10b can have a plurality of cooperating straps S (shown in FIGS. 13 and 17A, 17B, 18-20 in various exemplary configurations and cooperating sets) that attach to provide the brace 10 and hold the shoulder support member 40′ in anterior position over a glenohumeral joint. The straps S can include at least one torso strap 30 (e.g., chest or waist band) and at least one arm band strap 70 (e.g., a bicep band). As shown, the shoulder support member 40′ can directly attach to at least two of the different straps at the anterior side of the glenohumeral shoulder joint. The brace 10′ can include an optional restraint strap 34 for limiting range of motion. The brace 10′ can also be configured to position the shoulder support member 40′ over the posterior side of a target glenohumeral shoulder joint (FIG. 26). The brace 10, 10′, 10″ can also be configured to provide bilateral shoulder support (FIGS. 25, 29A).

As discussed above, the shoulder support member 40′ can include two attachment segments 46, 48 that attach to defined straps or strap segments, typically to at least two back straps 20, 124. The back straps 20, 124 may be provided as two or more separate straps or one long strap. One back strap 20 can be an over the shoulder strap and another can be an under the shoulder strap 124, each of which can attach to a torso section of the brace body on one end and to the shoulder support member 40′ on the other.

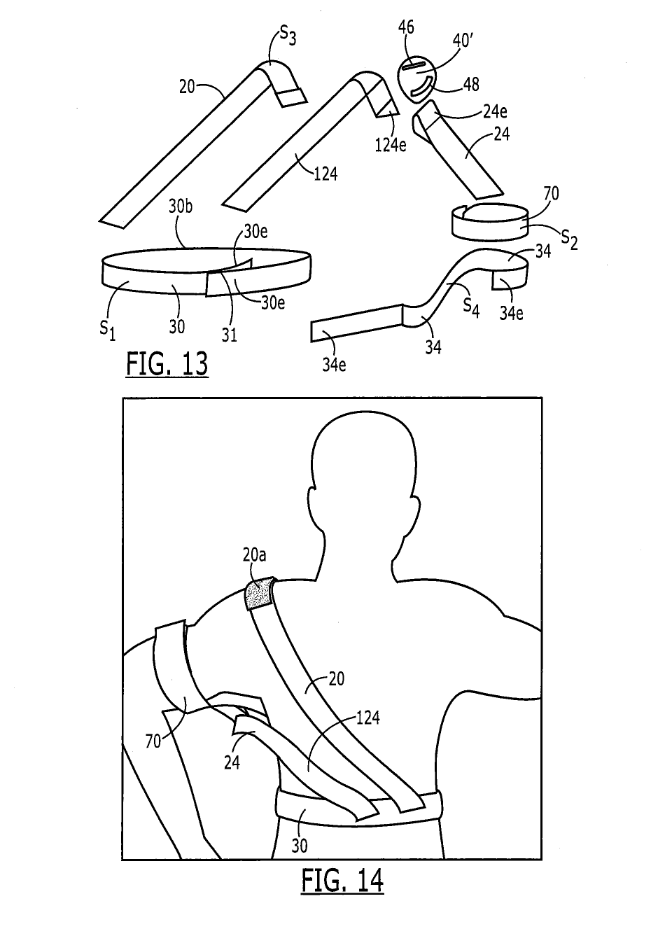

FIGS. 14-16, 21 and 22, show, for example, that the brace body 10b may include only one primary torso strap 30. The strap 30 may be semi-elastic or non-elastic, with adjustability to allow the device to be changed in size as required by the user while still providing sufficient anchoring or structural positioning. The torso strap 30 may contain anti-slide material such as a silicone, rubber or other suitable lining or surface features on the interior surface of the strap 30 to facilitate positional stability, such as by sticking to the user's skin or shirt. The anti-stick material is optional and, where used, may be optionally woven into or affixed to the strap. The torso (e.g., mid-chest or waist) strap 30 and back straps 20, 124 cooperate with the shoulder support member 40′ to provide compression and counter-force on the shoulder joint.

Referring to FIGS. 11-13, for example, the upper attachment segment 46 can include a planar and laterally extending slot 46s that receives an over the shoulder back strap 20 that extends upward from a back side of a torso strap 30b and the lower attachment segment 48 can receive an under the shoulder back strap 124 that extends upward from the back side of the torso strap 30b (FIGS. 14, 15, 21, 22). The upper slot 46s, where used, may have a smaller lateral dimension or width relative to the lower slot 48. The lower slot 48, where used, may be curvilinear or arcuate. Other attachment configurations of the straps S to each other and/or the shoulder support member 40′ may be used such as rivets, staples, hook and loop material (e.g., VELCRO® fasteners), adhesives, bonds, sewn fixations, combinations of same, or any other suitable attachment configuration(s) may be used.

The shoulder support member 40′ can be semi-rigid and malleable, e.g., a thermoplastic device that can be shaped to conform to a target anterior glenohumeral joint (e.g., anterior or posterior side) of a respective patient/user.

FIG. 12 shows that the torso strap 30 (e.g., mid-chest, or mid-body strap) can surround a torso (e.g., waist or above the waist chest region) with a width typically between 0.5 inches and 3 inches. The torso strap 30 can be a single elongate strap or a series of attached straps to form a closed loop configuration. Although shown as a single torso strap 30, a plurality of vertically stacked, side-by-side or spaced apart torso straps may be used (not shown). The torso strap 30 can have a linear elongate shape that can be attached to form the closed, substantially circular shape. The torso strap 30 can have free ends 30e that overlap to attach together at attachment zone 31. The attachment zone 31 can comprise hooks and loops, e.g., VELCRO® fasteners, but other attachment configurations are contemplated. The torso strap 30 can have an adjustable interior channel size. The end portions of the torso strap 30 can be attached via buckles, snaps, or other fittings. The torso strap 30 can attach to the back straps 20, 124. The torso strap 30 can also attach to the optional restraint strap 34 that can extend between the front of the torso strap to the arm band 70.

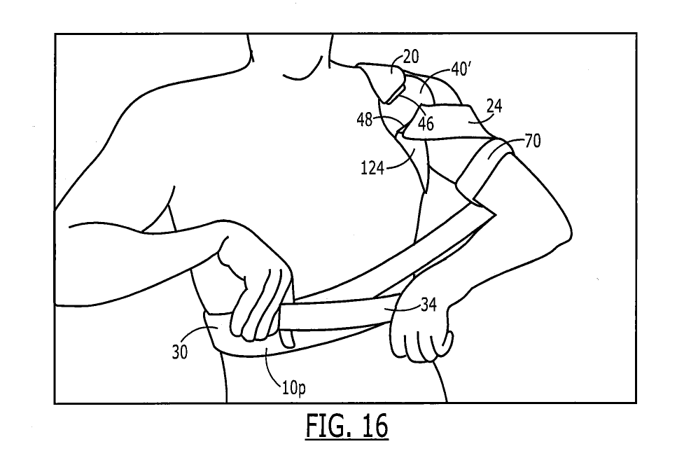

As shown in FIG. 13, the restraint strap 34 can have one end portion 34e that attaches to the torso strap 30 and another free end portion that attaches to the arm band 70. In use, the restraint strap can be attached to the arm band 70 so that it forms a curved portion 34c that typically extends around a major portion of a perimeter of the arm band 70. FIG. 16 illustrates that the arm band 70 and restraint strap 34 can be a single strap. Where used, the restraint 34 can limiting a range of motion relative to the brace and user without such a feature.

As shown in FIGS. 12-15, 21 and 22, the arm band 70 can include at least one arm strap 24 that rises from the arm band 70 and attaches to another portion of the brace body 10b, typically to the shoulder support member 40′. The arm strap 24 of the brace is preferred to be semi-elastic or non-elastic.

The term “semi-elastic” with the word “strap” or “band” is used in a textile manner to describe that the member is slightly elastic so as to be able to elastically elongate, stretch and/or change in length, but is configured to have a stretch that is less than 100% so that it cannot double its length. For example, for an elastic strap with 100% stretch it can elongate from an initial length of, for example, 4 inches to a fully elongated length of 8 inches. In preferred embodiments, a “semi-elastic” strap has a stretch that is in a range of 10%-80% typically between about 70% and 20%, and more typically between 60% and 40% stretch, including about 60%, about 55%, about 50%, about 45% and about 40% stretch. One example of a suitable strap material is a woven polyester elastic, style 6157, from Elastic Cord and Webbing, Inc., Dundee, Ill. (theecwcorp.com), stretch 60. Different straps may have different stretch or the same stretch.

When the brace 10′, 10″ is on a user, the strap 20 that goes over the back, and under the strap or strap segment 124 that goes under the armpit are both stretched, and as the arm moves into abduction and external rotation, the elastic straps 20, 124 retract, pulling the shoulder piece 40′ posterior to provide the compression against the glenohumeral joint. Stretch occurs as the person moves their arm.

The arm band 70 can be sized to fit on the upper arm of the user. The arm band 70 may be a band formed of semi-elastic or non-elastic material, with adjustability in its useful size to allow for bicep flexion and extension. Like the torso strap 30, the arm band may contain an anti-slide material or feature(s) such as a silicone lining on the interior of the band 70 to facilitate positioning and facilitate sticking to the user's skin or shirt. The anti-slide feature is optional, and may be woven into, formed on or attached to, the band 70 where included. Additionally, the arm band 70 can have an attachment region to allow for optional front restraint strap placement onto the arm band. The arm band of the brace may be formed of semi-elastic or non-elastic material, and is preferred to adjustable in usable length. The arm strap 24 may be connected to the arm band 70 and may be connected to the shoulder component 40′ in any suitable manner. The arm strap 40 can cooperate with the semi-rigid shoulder support member 40′ to provide compression and counter-force on the shoulder joint.

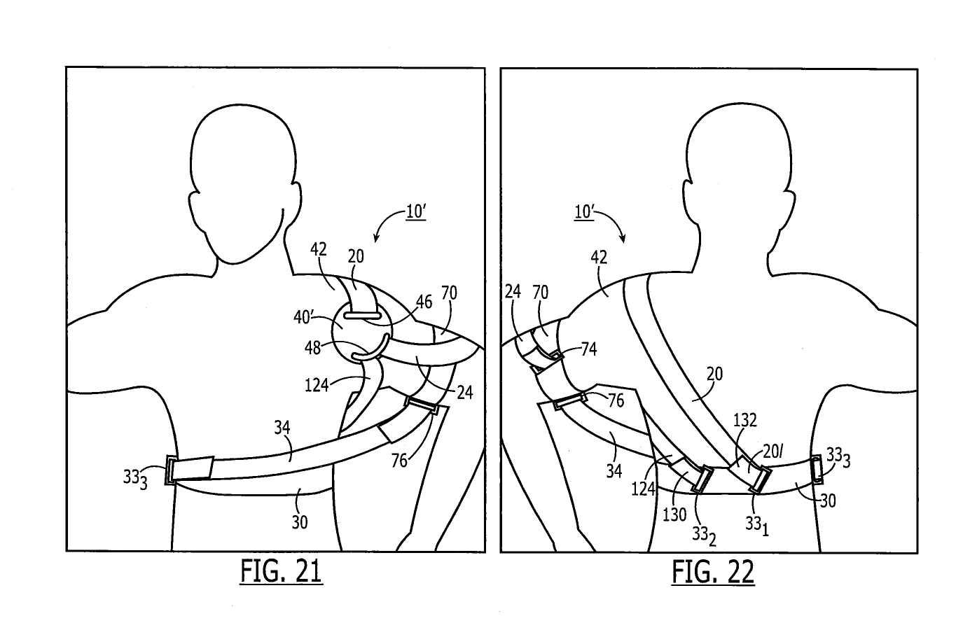

The useable length of the arm strap 24 is preferably, but optionally, adjustable. The strap 24 and arm band 70 can allow for optional front restraint strap placement on the arm band 70. The attachment may employ hook and loop material, or another mechanical fastener, such as a snap, rivet or buckle, or may comprise an adhesive. The arm strap 24 is preferred to be semi-elastic or non-elastic. The useable length may be adjustable. The arm strap 24 can attach to a front or outer perimeter side of the arm band 70 (FIGS. 12, 21).

The arm band 70 can have releasably attachable opposing ends providing for an adjustable length/size. The arm band 70 can also be provided as a closed continuous elastic band of sufficient width that can accommodate different size arms of different users. The arm band 70 can be provided in different sizes, for different users, e.g., XS, S, M, L, XL and the like.

The arm band 70 can be resilient and sized and configured with elasticity sufficient to stretch to surround and reside snugly about and against the desired anatomical object held therein. The arm band 70 can be fabricated to provide elasticity or stretch in the lateral direction sufficient to provide a desired support structure for the arm strap 24 and/or optional restraint strap 34. In certain embodiments, the arm band 70 can be configured with anisotropic (two-way) elasticity or stretch, so that the stretch in the lateral direction is generally greater than in the longitudinal direction.

The arm band 70 can be formed of a breathable air-permeable fabric that inhibits sweat accumulation and can flex with the movement of the patient (flexure of the muscles thereunder and/or flexure attributed to movement of the arm) so as to promote patient comfort. As such, the arm band 70 can be configured with sufficient stretch to firmly reside on the patient while not imparting undue compression force onto the arm or other body part of the subject. In certain applications, the arm band 70 may inhibit bruising or “pinching” of the skin and/or tissue of the user. The arm band 70 can be configured to surround the limb in a snug, tight, form-fitting conformable configuration that is functionally “molded” in situ to take the shape of the surround arm of the user. For example, in position, the arm band 70 may be resiliently altered in shape from its non-use configuration into a substantially cylindrical (for substantially straight limb anatomical physiological structure) or into a substantially frusto-conical shape (for limbs that taper from larger to smaller cross-sectional widths top to bottom). Examples of suitable breathable fabrics include synthetic “stretch” or elastomeric fibers. An example of a stretch fiber is a synthetic manufactured fiber, such as spandex, in which the fiber forming substance is a long-chain synthetic polymer.

As shown in FIGS. 13 and 24, the upper end portion 24e of the arm strap 24 can attach to the lower attachment segment 48 of the support member 40′. Also, in some embodiments, an upper end portion 124e of the back strap 124 and an upper end of the arm strap 24 can both reside in the attachment slot 48s. While the slot 48s may be a continuous length as shown. Two separate, typically adjacent, slots may be used, one for each strap end portion 24e, 124e (not shown). Again, other attachment configurations may be used.

The torso strap 30 can comprise additional loop 8 and loop 10. Loop 8 and loop 10 may be positioned diagonally relative to the waistband as shown. Another loop 12 which may be a vertical loop may be present on the waistband.

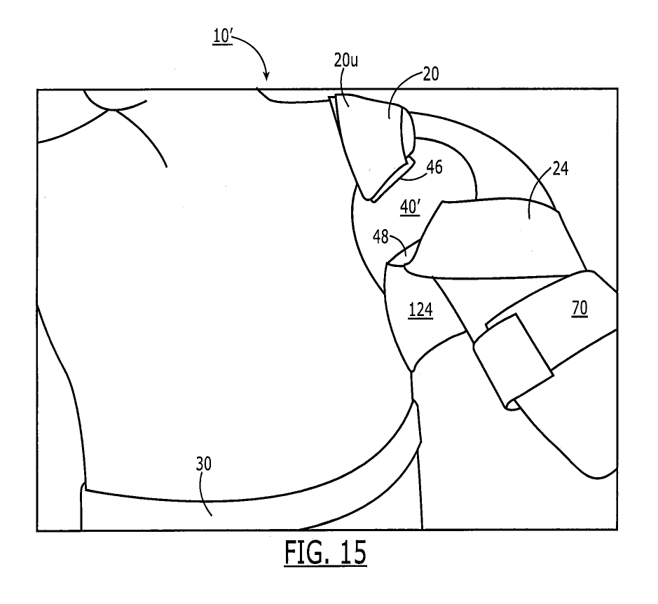

FIGS. 14 and 15 shows that the upper end portion 20u of the over the shoulder back strap 20 can releasably attach to the shoulder support member 40′. FIGS. 23 and 24 show that the upper end 20u can be (non-detachably) affixed to the shoulder support member 40′.

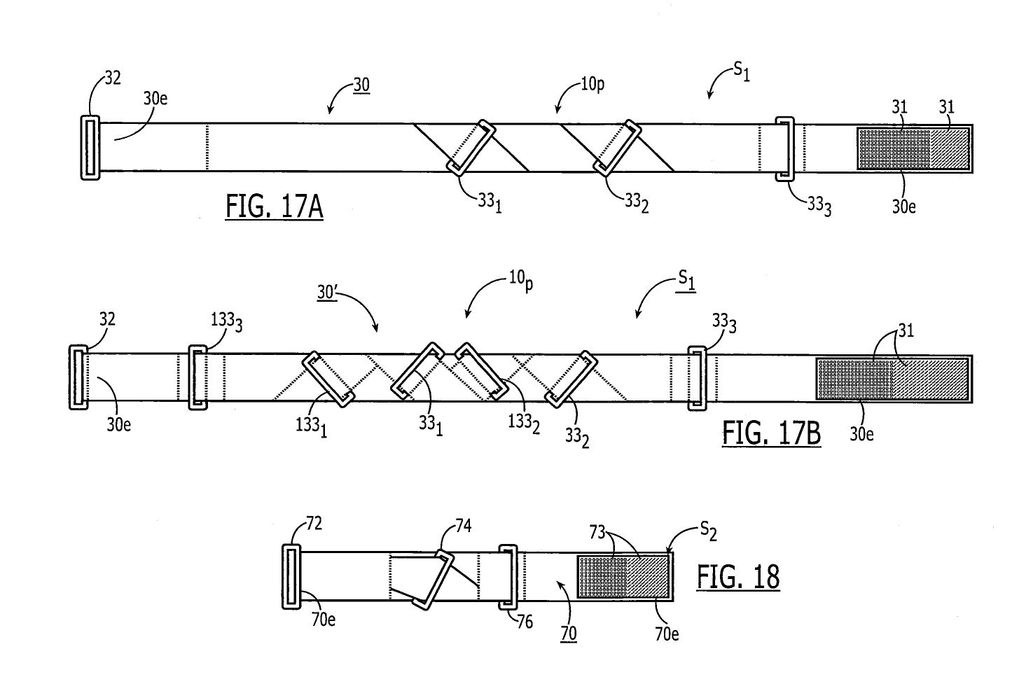

FIG. 17A shows an example of a torso strap 30 (S₁) with end portions 30e having attachment members or features 31, 32. FIG. 17B illustrates a bilateral version of the torso strap 30′ that can be used to attach to two shoulder support members 40′, one on a left side and one on a right side. FIG. 25 illustrates an assembled bilateral shoulder brace configuration.

As shown in FIGS. 17A and 17B, one end portion 30e of the torso strap 30 can include hook and loop fastener features 31 that can extend through a loop attachment member 31 on the other end 30e. The torso strap 30 can include a plurality of additional loop attachment members 33.

FIG. 17A illustrates three loop attachment members 33₁, 33₂, 33₃. As shown, two of the loop attachment members 33₁, 33₂ can be diagonally oriented and the other 33₃, typically located closer to one of the end portions 30e relative to the diagonally oriented members, can be vertical. The diagonal members 33₁, 33₂ can hold/engage a respective back strap 124, 20. The other attachment member 33₃ can engage the optional restraint 34. The torso strap 30 can be flipped to accommodate straps 20, 124, for right or left shoulder brace configurations.

As noted above, FIG. 17B illustrates another version of the torso strap 30′ and shows a second pair of the diagonal loop attachment members 133₁, 133₂. The first pair of (parallel) diagonal attachment members 33₁, 33₂ can connect to back straps 20, 124 that extend up and to the left shoulder and the second pair of diagonal loop attachment members 133₁, 133₂ can currently or alternatively connect to back straps 20, 124 that extend up and to the right shoulder (FIG. 25). FIG. 17B also illustrates a second vertical loop attachment member 133₃ for an optional additional restraint strap. The torso strap 30′ shown in FIG. 17B may be particularly suitable for bilateral use. Thus, the bilateral version may include four (4) diagonal loops on the torso band connecting two sets of straps 24, 124 and respective shoulder support members 40′.

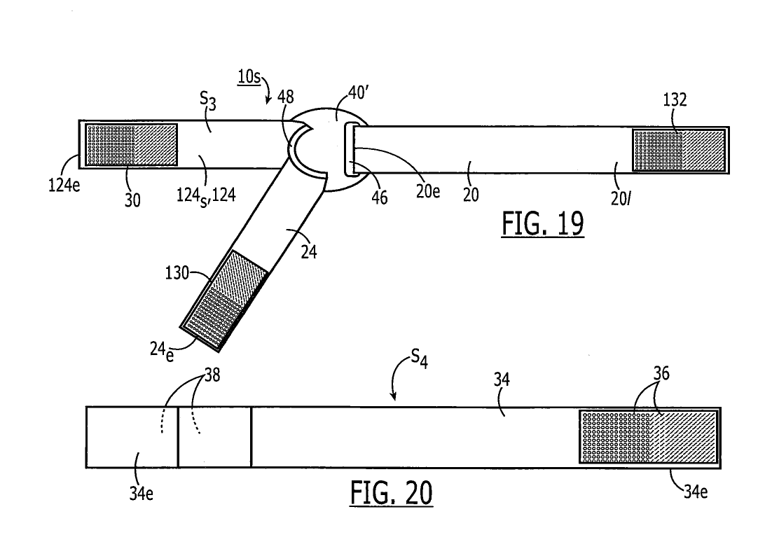

FIG. 18 shows an example of an arm band 70 (S₂) according to embodiments of the invention. The arm band 70 can include attachment members 72, 73 to connect opposite end portions of the arm band 70e. In the embodiment and shown, one attachment member 72 comprises a vertical loop and the other 73 comprises hook and loop material. The arm band 70 may optionally also include a diagonally or angled attachment loop 74 positioned along its length interior to the outer ends 70e, and a vertical attachment loop 76 laterally spaced apart from the angled attachment loop 74 positioned along its length, with the vertical attachment loop 74 typically positioned closer to the hook and loop material 73, where used, and the diagonal attachment loop positioned closest to end loop 72. The arm band ends 70e can attach to form the arm band and can be positioned about the arm of the wearer. The arm band 70 is connected to additional elements of the stabilizing brace 10 as discussed herein.

FIG. 19 shows a shoulder support strap sub-assembly 10s. The shoulder support sub-assembly 10s can hold a strap S₃ that can form both a segment 124s of or the entire under the shoulder back strap 124 and the arm strap 24. The strap segment 124s or strap 124 and arm strap 24 may also or alternatively be provided as separate straps. The sub-assembly 10s can also include a portion of or the entire the over the shoulder strap 20. The sub-assembly 10s can have three strap legs or leg segments attached to the shoulder support member 40′. Each leg of the strap S₃ or straps 24, 124 and the leg end portion 20e of the strap 20 may be permanently attached to the shoulder support member 40′. Arm strap 24 can have a different length, and is typically shorter than, the strap segment 124. Strap 24 and strap and/or strap segment 124, 124s can attach at different, typically adjacent, regions of the shoulder support member 40′.

Strap 24 can be affixed to the shoulder support member 40′, typically, but optionally, through a lower slot 48s in the shoulder support member 40′. The free end of strap 24 and strap or strap segment 124 or 124s can have an attachment feature such as a fastener or connector, which is preferred to be hook and loop material 130, which is used to connect the respective straps 24, 124/124s to other elements of the device 10′. One end 20e of the strap 20 can be permanently attached at one end to the shoulder support member 40′, such as through slot 46 above of the lower slot 48 of the shoulder support member. The opposite end of the strap 20/extends away from the shoulder support member 40′. The end of strap 20/that extends away from the shoulder support member 40′ can include an attachment feature such as a fastener or connector 130 for connecting the shoulder support member 40′ to other elements of the stabilizing brace. In a preferred embodiment, the attachment member 132 comprises hook and loop fastener material as shown.

FIGS. 23 and 24 show that the strap sub-assembly 10s can be provided as an integral unit for use in a heated water bath 140 to heat the member 40′ to be malleable to form the shoulder support member 40′ in a custom fit for a particular user (FIG. 23).

FIG. 20 shows another strap S₄, which is the optional limiting strap 34. The optional limiting strap 34 can have an attachment feature of fastener such as a connector at each end 34e of the optional limiting strap 34. The fastener or attachment features can comprise hook and loop fasteners 36, 38. The optional limiting strap may be connected to the torso band 30 at one and to the arm band 24 at the opposite end.

As shown in FIGS. 21 and 22, the diagonal loops 33₂, 33₁, can be positioned at the back of the user, and the vertical loop 33₃ can be positioned opposite the injured or unstable shoulder 42.

FIG. 26 illustrates a shoulder brace for posterior shoulder support according to embodiments of the present invention.

To don the brace 10′ the torso band 30 can be positioned at about mid-chest and under the pectorals, typically with end portions thereof secured together to form a closed configuration, such as with the fasteners 31, 32. The arm band 70 can be attached to and/or positioned about the arm of a user. The diagonal loop 74 is preferred to be positioned on the back side of the arm that is adjacent to the injured or unknown stable shoulder. Loop 76 can be positioned to be adjacent to the user's body, when the arm is hanging at the users side. The arm band 70 or torso band 30 can be positioned on a user first.

With the torso band 30 and the arm band 70 secured to the user's body, the shoulder support assembly 10s can be attached to the arm band 70 and to the torso strap/band 30. End 20l of strap 20 can be attached to a diagonal loop 33 of the torso strap 30. End 124e of strap 124 can be attached to the other diagonal loop 33 of the waistband while the end 24e of the arm strap 24 can be attached to the diagonal loop 74 on the armband.

The optional limiting strap 34 may be connected at one and to the vertical loop 331 on the torso band 30, and at the opposite end to the vertical loop 76 on the arm band 70.

Thus, some embodiments are characterized by a brace body 10b having various straps S and attachment zones, an arm band 70 (e.g., a bicep band) with an arm strap 24 attached thereto, and a semi-rigid shoulder support member 40′ that is attached to, cooperates with and/or is in communication with various straps S at the shoulder joint with an optional restraint to limit range of motion.

The shoulder support member 40′ can have a shape and size to fit (only) the anterior portion of the shoulder. This shoulder member 40′ may optionally be customized by a variety of methods, moldable by a variety of materials and/or methods of molding, which may vary from rigid to flexible and soft. The shoulder member 40′ may be attached to the torso strap 30 and back straps 20, 124 by a variety of methods that include, but are not limited to, slots within the member 40′. The shoulder member can cooperate with the brace body 10b, e.g., straps S to provide a counter-force on the anterior of the shoulder joint. The shoulder member 40′ may be any form or shape as discussed above with respect to the first embodiment 40, that adequately covers the anterior or posterior portion of the shoulder, so as to provide compression and counter-force while the user moves his or her arm.

In some embodiments, in use, the user obtains compression and counter-force from the shoulder component by the attached torso, back, and arm straps, 30, 20, 124 and 24 to the shoulder member 40′. By providing tension on the shoulder support member 40′, the various straps are able to hold the shoulder member 40′ in place and work together to provide a counter-force against the shoulder joint.

When the arm is at a neutral position, the two back straps 20, 124 are in tension pulling the shoulder member 40′ posteriorly. The two back straps 20, 124 attach at different points to the shoulder member 40′ providing an even distribution of the posterior force across the head of the humerus. As a user begins to abduct his or her arm, the back strap 20 that is attached to the upper portion of the shoulder component 40′ retracts, while a second back strap 124 attached to the lower portion of the shoulder component 40′ lengthens. The change in lengths of both back straps 20, 124 can provide the counter-force to compress the shoulder member 40′ against the shoulder. The change in lengths of the back straps 20, 124 due to tension applied can be greater the higher the arm is abducted. Greater change in the lengths of the straps 20, 124 results in a greater compressive force provided to the shoulder. The arm band 70 and arm strap 24 may be adjusted to limit external rotation and abduction while providing an additional counter force on the shoulder support member 40′. The arm band 70 and/or arm strap 24 can help to slightly rotate the shoulder support member 40′ with the humeral head as the arm is in abduction and external rotation. The device 10 provides a functional shoulder brace providing compressive support of the shoulder, such as at the glenohumeral joint.

The shoulder brace provides compression, such as compression of the glenohumeral joint, while allowing for functional range of motion after a shoulder injury.

The shoulder component may sit above the glenohumeral joint.

The device allows for functional movement while wearing the brace, and provides counter-force compression for the shoulder, such as at the glenohumeral joint. The device limits the allowed range of motion, and may be adjusted, depending upon the degree of instability of the shoulder.

The shoulder support member 40′ is preferred to be custom molded to the user. Custom molding may be accomplished by taking measurements of the users shoulder at the point of application of the shoulder support member to the user, and forming the shoulder support member at a medical device laboratory. However, in a preferred embodiment, shoulder support member 40′ is formed of a material such as a plastic that softens when heated, and which may be formed and reformed to a desired shape while softened. Heating of the shoulder support member may be accomplished by placing the shoulder support member in container of heated water 140 (FIG. 23).

As discussed above with respect to embodiment 40, in some embodiments, the shoulder support member 40′ is capable of deformation and reformation when heated to a temperature of about 150° C. This permits the shoulder support member 40′ to be formed anywhere that hot water of the required temperature is available.

While the shoulder member 40′ is elevated to a temperature sufficient for formation of its shape, the member is placed on a center of an injured or unstable shoulder 42 of a user and in the position shown in FIG. 24, and formed to the shoulder. The shoulder support member 40′ can be allowed to cool after formation to retain the desired shape.

Straps 20, 124, 24, 30, 70 may be elastic or non-elastic, or a combination of elastic and inelastic straps. Typically, at least over and under the shoulder straps 20, 124 are elastic, and may be semi-elastic. The connector or fastener may be hook and loop material to allow for attachment of the ends, and adjustability of the length of the straps to fit the anatomy of the individual user. Other connectors that permit adjustment of the length, such as buckles, may be used to form connects ends of straps to form the device.

The hook and loop fastener material described above for various attachments may also include or be replaced with equivalent connectors, such as snaps, rivets, buckles and adhesive to connect the straps as required to form the device.

Some of the straps S may comprise a non-stick material or lining on the interior of the strap to facilitate positioning and retard sticking to the user's skin or shirt. The non-stick material may be a silicone material, Teflon, PTFE or similar material. The non-stick material lining is optional, and may be woven into, coated on, or affixed to, the straps.

The user obtains compression and counter-force from the shoulder component by attaching the chest, back, arm straps to the shoulder component as described above. The straps of the device hold the shoulder support member in place and provide tension on the shoulder support member, which provides a force placed against the shoulder joint to stabilize the shoulder joint.

When the arm is in a neutral position, the back straps 20, 124 are in tension pulling the shoulder support member posteriorly. The two back straps attach at different points to the shoulder support member 40′ to distribute of the posterior force across the head of the humerus.

As a user begins to abduct his or her arm, strap 20 retracts, while strap 124 lengthens. As the user abducts and externally rotates his or her arm, straps 124 and 20 work together with the shoulder support member, arm band, and waist band to provide compressive support. When the brace is assembled and applied, straps 20 and 124 are put in tension, and as the user moves their arm, the tension wants to lessen, which pulls the support piece posteriorly, although strap 124 may not pull the support piece posteriorly.

The relative change in lengths of the straps provides a force to compress the shoulder support member against the shoulder. The change in lengths of the back straps is greater the higher the arm is abducted. Greater change in the lengths of the straps results in a greater compressive force provided to the shoulder. The arm band and the end 24e of strap 24 may be adjusted.

Greater external rotation limitation is achieved by having the user place his or her arm in an internally rotated position and adjusting the strap 24 to tighten the strap, and provide additional counter force on the shoulder component. The arm band helps to slightly rotate the shoulder support member with the humeral head as the arm is in abduction and external rotation. The device provides a functional shoulder brace providing compressive support of the shoulder, such as at glenohumeral joint.

The shoulder brace provides compression, such as compression of the glenohumeral joint, while allowing for functional range of motion after a shoulder injury. The shoulder component may sit above the glenohumeral joint. The device allows for functional movement while wearing the brace, and provides counter-force compression for the shoulder, such as at the glenohumeral joint. The device limits the allowed range of motion, and may be adjusted, depending upon the degree of instability of the shoulder.

FIGS. 27A, 27B, 28A, 28B, 29A and 29B illustrate that the brace 10″ can include straps S that attach to segments of a brace body 10b that is configured with a torso portion 10p that can provide torso attachment features 130 that can function similar to the torso strap 30 and that can include a sleeve that can provide at least one arm strap attachment feature 170 that function similar to the arm band 70 (FIG. 12, 21).

The brace body 10b can include the torso portion 10p that has a section with attachment features 130. The attachment features 130 can reside above the waist, typically mid-chest, and can comprise the connectors described with respect to the strap 30 and/or hook and/or loop like fasteners (e.g., VELCRO) or other configurations or strap attachment features. The attachment features 130 can extend totally or partially laterally about the torso section or portion 10p to allow for user-to-user strap positional and/or alignment adjustability. As shown, the torso attachment features 130 can extend about both a front and back part of the brace body 10b to allow the brace body to be used for posterior and/or anterior glenohumeral shoulder support. However, the attachment features 130 can extend about only a front or only a back portion in a continuous or discontinuous manner. The attachment features 130 can be provided in a continuous straight (circular) line as shown or provided as more localized right or left side features, for example, and some attachment features can reside above or below others for attaching to a defined strap segment, typically to both an over and under the shoulder strap or strap segment 20, 124, each of which can also attach to the shoulder support member 40′. The attachment features 130 can be provided in the form of a belt or cooperating strap 30 that can be used with the torso portion 10p.

The brace body 10b can also include at least one arm segment 10a that can be attached to the torso segment 10p and can provide an arm strap attachment feature 170 typically at a bicep or lower location of an arm of a user. The arm strap attachment feature 170 can be hook and/or loop fasteners (e.g., VELCRO) that can extend totally or partially laterally about the arm portion 10a to allow for user-to-user strap positional and/or alignment adjustability. As shown, the arm strap attachment feature 170 can extend about both a front and back part of the arm 10a to allow for posterior and/or anterior glenohumeral shoulder support. However, the attachment feature 170 can extend about only a front or only a back portion in a continuous or discontinuous manner. The attachment feature 170 can be provided in a straight (circular or semi-circular) line as shown or provided as more localized right or left side features, for example. The arm attachment feature 170 can engage the arm strap or strap segment 24 that can also be attached to the shoulder support member 40′. The arm attachment feature 170 can be any suitable connector or attachment configuration and is not limited to hook and/or loop fastener materials (e.g., VELCRO).

The shoulder support member 40′ can be attached to the three straps or strap segments, 20, 124 and 24. The support member 40′ can be held in a pocket 10 attached or formed in the brace body 10b. The shoulder support 40′ can be or otherwise held against or by the brace body 10b. No physical attachment to the brace body is required. Where used, the pocket 15 can be a recess or material compartment. In some embodiments, the location for the shoulder support member 40′ can have visual indicia 15i to allow a user to adjust the straps 20, 124 so that the shoulder support member 40′ resides over the indicia 15i to be positioned properly. The visual indicia 15i may be a color-coded region and/or have text and/or or stylized or defined shape, for example.

In some embodiments, the shoulder support member 40′ is releasably attached to the brace body 10b via hook and loop fasteners, adhesive and/or a pocket 15. Where used, the pocket 15 can be an internal or external pocket of the same material as the brace body or may be a different material. The pocket 15 may have a closed outer surface or comprise mesh material, for example.

In some embodiments, the shoulder support member 40′ is attached to both the brace body 10b and the straps or strap segments 20, 124 and typically also attached to the arm strap 24. In some particular embodiments, the shoulder support member 40′ is fixedly attached to straps or strap segments 20, 124, 24 and releasably attached (detachably coupled to) to the brace body 10b or releasably held (detachably coupled to) in a pocket 15 of the brace body 10b.

The brace body 10b can have a shirt-like configuration with sleeved-arms 10a and a torso portion 10p. The shirt-like configuration may be a “T-shirt” like configuration with a crew or “V-neck” opening to allow a user to slip the shirt part of the brace 10″ over his or her head. Also, while shown as including two arms 10a, the brace body may include a single arm 10a as shown in FIG. 1, for example. This may be particularly true for dedicated left or right side shoulder brace configurations, for example.

In some embodiments, the brace body 10b may be sleeveless and/or may be configured to provide at least one laterally extending integrated arm band segment (circular laterally extending segment) attached to the torso portion 10p.

While shown as a unitary, single-piece “slip on” shirt-like body, the shirt-like body can have segments that can be releasably attachable via zippers, buttons, snaps, hook and loop fasteners or other features.

The shoulder support member 40′ is preferably semi-rigid and a moldable or malleable member as described above, but other shoulder support configurations/members may be used. Further, in some embodiments, the brace body 10b and straps S may provide suitable support or protection without requiring the semi-rigid support member 40′.

The brace body 10b and associated straps S can be provided in different sizes and may be provided in gender variations and adult and children sizes. The brace body 10b can be provided in a snug or compression or “stretch to fit” type material for anchoring the ends of certain straps S at the torso and/or arm attachment segments 130, 170. The torso segment can cooperate with a belt (not shown) to help secure the torso segment in position on a user for positional stability while the shoulder is able to move.

Although shown as providing both right and left anterior configurations with the attachment features 130, 170, the brace body 10b can be provided in dedicated use configurations, e.g., only right anterior (with only right arm attachment features 170), only left anterior (with only left arm attachment features 170), only front bilateral anterior configurations and only bilateral posterior versions (each not requiring part of the torso attachment features).

In summary, it is contemplated that the shoulder braces 10, 10′, 10″ can be used for posterior, anterior and/or bilateral (anterior and/or posterior) shoulder supports. FIGS. 28A and 28B illustrate a posterior use of the shoulder brace 10′. FIGS. 29A and 29C illustrate an exemplary bilateral use. The brace 10, 10′, 10″ can be applied anteriorly and posteriorly, as well as on both shoulder simultaneously either as only a series of cooperating straps ors incorporated into a compression/performance wearable garment such as a shirt, T-shirt, jacket, or vest-like device.

Similar to the embodiment discussed above with respect to FIG. 21, for example, the shoulder brace 10″ provides compression and counter-force from the shoulder component 40′ by attaching the under the arm chest strap 124, the over the shoulder back strap 20, and the arm strap 24 to the shoulder component 40′ as described above. The straps 20, 124 and/or brace body 10b (e.g., pocket 15) hold the shoulder support member 40′ in place and provide tension on the shoulder support member 40′, which provides a force placed against the shoulder joint to stabilize the shoulder joint.

When the arm is in a neutral position, the back straps 20, 124 are in tension pulling the shoulder support member 40′ posteriorly. The two back straps 20, 124 can attach at different points to the shoulder support member 40′ to distribute of the anterior or posterior force across the head of the humerus.

As a user begins to abduct his or her arm, strap 20 retracts, while strap 124 lengthens. As the user abducts and externally rotates his or her arm, straps 124 and 20 work together with the shoulder support member 40′, arm 10a via arm fasteners 170, and torso portion 10p via attachment features 130 to provide compressive support. When the brace 10″ is assembled and applied, straps 20 and 124 are put in tension, and as the user moves their arm, the tension wants to lessen, which pulls the support piece 40′ posteriorly (for anterior support), although strap 124 may not pull the support piece posteriorly.

The relative change in lengths of the straps provides a force to compress the shoulder support member against the shoulder. The change in lengths of the back straps is greater the higher the arm is abducted. Greater change in the lengths of the straps results in a greater compressive force provided to the shoulder. The arm strap 24 length between the shoulder support member 40′ and arm fasteners 170 may be adjusted.

Greater external rotation limitation is achieved by having the user place his or her arm in an internally rotated position and adjusting the strap 24 to tighten the strap, and provide additional counter force on the shoulder component. The arm attachment features 170 can help to slightly rotate the shoulder support member 40′ with the humeral head as the arm is in abduction and external rotation. The shoulder brace 10″ provides a functional shoulder brace providing compressive support of the shoulder, such as at glenohumeral joint.

Again, the shoulder braces provide compression, such as compression of the glenohumeral joint, while allowing for functional range of motion after a shoulder injury. The shoulder component 40′ may sit above (external to or over) the glenohumeral joint. The shoulder brace 10, 10′, 10″ allows for functional movement while wearing the brace, and provides counter-force compression for the shoulder, such as at the glenohumeral joint. The device limits the allowed range of motion, and may be adjusted, depending upon the degree of instability of the shoulder.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

That which is claimed:

1. A shoulder brace, comprising:
a plurality of straps; and
a semi-rigid shoulder support member, wherein the semi-rigid shoulder support member has a user-specific custom shape that substantially conforms to an anterior or posterior of a glenohumeral joint of a shoulder of a respective user, and wherein, with the shoulder brace in use, the semi-rigid shoulder support member is sized and configured to reside over the anterior or posterior of the glenohumeral joint of the shoulder of the user.

2. The shoulder brace of claim 1, wherein the straps include (i) at least one over a shoulder strap and (ii) at least one under the shoulder strap, and wherein the over and under the shoulder traps are attached to the shoulder support member at spaced apart portions of the shoulder support member and to a torso strap or fasteners on a torso portion of a brace body and are configured to extend and retract in a cooperating manner to apply compression against the shoulder support member to hold the shoulder support member against the anterior or posterior of the glenohumeral joint during movement of an arm that is adjacent the shoulder support member.

3. The shoulder brace of claim 1, wherein the straps include (i) a first elongate strap that is attached to an upper portion of the semi-rigid shoulder support member and (ii) a second elongate strap that is attached to a portion of the semi-rigid shoulder support member below the first strap, wherein the first strap and the second strap are also attached to a torso strap or fasteners on a torso portion of a brace body, and wherein the first and second straps are configured to extend and retract in a cooperating manner to apply compression against the semi-rigid shoulder support member to hold the semi-rigid shoulder support member against the anterior or posterior of the glenohumeral joint during movement of an arm that is adjacent the semi-rigid shoulder support member.

4. The shoulder brace of claim 1, wherein the shoulder support member has a thickness between about 0.125 inches to about 0.25 inches and has a plurality of vertically spaced apart slots, including a first upper slot that engages an over the shoulder back strap and a second lower slot that engages at least a second strap.

5. The shoulder brace of claim 1, wherein the semi-rigid shoulder support member is a thermoplastic shoulder support member.

6. The shoulder brace of claim 1, wherein the plurality of straps comprises a torso strap, that, in use, defines a closed shape to surround a torso segment of a user at or above a waist of the user, an over a shoulder strap, an under the shoulder strap, and at least one arm strap, and wherein the over and under the shoulder straps and the arm strap are attached to the shoulder support member, and wherein the over and under the shoulder straps are also attached to the torso strap while the arm strap is attached to an arm band and/or the torso strap.

7. The shoulder brace of claim 1, further comprising a brace body with at least one sleeve and a torso portion, the sleeve having a region with a strap attachment segment and the torso portion having a strap attachment segment.

8. The shoulder brace of claim 7, wherein the plurality of straps comprises an over a shoulder strap, an under the shoulder strap, and at least one arm strap, and wherein the over and under the shoulder straps and the arm strap are attached to the semi-rigid shoulder support member, and wherein the over and under the shoulder straps are also attached to the torso attachment segment while the arm strap is also attached to the arm attachment segment.

9. The shoulder brace of claim 7, wherein the brace body comprises left and right side sleeves, each with a strap attachment segment sized and configured to reside proximate a bicep portion of a respective sleeve, wherein the right side strap attachment segment attaches to a right side arm strap for a right side anterior or posterior shoulder brace configuration, and wherein the left side strap attachment segment attaches to a left side arm strap for a left side anterior or posterior shoulder brace configuration.

10. The shoulder brace of claim 7, wherein the brace body has a shirt configuration that provides the torso portion and right and left arm sleeves, and wherein the brace body comprises right and left side pockets to releasably engage a respective semi-rigid shoulder member.

11. The shoulder brace of claim 1, wherein the at least one semi-rigid shoulder support member is attached to three strap segments including an over the shoulder strap segment, an under the shoulder strap segment and an arm strap segment.

12. The shoulder brace of claim 11, wherein the under the shoulder strap segment and the arm strap segment are attached to a lower end portion of the semi-rigid shoulder support member, and wherein the over the shoulder strap segment is attached to an upper end portion of the semi-rigid shoulder support member.

13. The shoulder brace of claim 11, wherein the under and over the shoulder strap segments and the arm strap segment are fixedly attached to the semi-rigid shoulder support member.

14. The shoulder brace of claim 2, wherein the shoulder brace has the torso strap, and wherein the torso strap comprises diagonally oriented, parallel loop segments that attach to the over and under the shoulder back strap segments.

15. A shoulder brace, comprising:
a shoulder support member;
a torso strap or torso portion of a brace body with a strap attachment feature;
an elongate over a shoulder strap comprising opposing longitudinally spaced apart first and second end portions, the first end portion connected to the torso strap or torso portion and the second end portion connected to the shoulder support member; and
an arm strap that is connected the shoulder support member and an arm band or sleeve of a brace body.

16. The shoulder brace of claim 15, further comprising an under the shoulder strap comprising opposing longitudinally spaced apart first and second portions, the first portion connected to the torso strap or torso portion of the brace body and the second portion connected to the shoulder support member at a location that is below the second end portion of the over the shoulder strap.

17. The shoulder brace of claim 15, wherein the shoulder support member is semi-rigid and comprises an upper laterally extending slot that engages the over the shoulder trap and a lower curvilinear slot that engages the arm strap and the under the shoulder strap.

18. The shoulder brace of claim 15, wherein the shoulder brace has a brace body that has a shirt configuration with at least one arm portion with a sleeve and a torso portion, wherein the at least one arm portion has a strap attachment feature for the arm strap, and wherein the torso portion has the torso strap attachment feature for the first end portions of the over and under the shoulder straps.

19. The shoulder brace of claim 18, wherein the brace body shirt configuration has right and left arm sleeves, and wherein the brace body comprises right and left side pockets to releasably engage a respective semi-rigid shoulder member.

20. The shoulder brace of claim 15, wherein the under and over the shoulder straps and the arm strap are fixedly attached to the shoulder support member.

21. The shoulder brace of claim 19, wherein the shirt configuration allows for anterior and posterior arrangements of the shoulder support member and/or bilateral arrangements of respective straps and shoulder support members.

22. The shoulder brace of claim 15, wherein the shoulder brace can be used for anterior and/or posterior positioning of the shoulder support member.

23. The shoulder brace of claim 1, wherein at least some of the straps are elastic with a stretch of between about 40% to about 60%.

24. A method of providing support to a shoulder of a user, comprising:
providing a shoulder brace with a semi-rigid shoulder support member that resides over an anterior or posterior glenohumeral joint of a user with a compromised shoulder; and
allowing a functional substantially full range of motion when the user wears the shoulder brace while providing compressive support to a glenohumeral joint of the compromised shoulder when the user wears the shoulder brace.

25. The method of claim 24, wherein the shoulder brace comprises a plurality of cooperating straps including an arm strap, an over the shoulder strap and an under the shoulder strap that are attached or attachable to the shoulder support member, and wherein the method further comprises:
attaching the arm strap to a sleeve of a brace body or to an arm band on an arm of a user adjacent a compromised and/or injured shoulder; and
attaching the over and under the shoulder traps to a torso strap or a torso portion of a brace body,
wherein the shoulder brace provides compressive support by extending and retracting the over and under the shoulder straps in a cooperating manner to apply the compression against the shoulder support member to hold the shoulder support member against the anterior or posterior of the glenohumeral joint during movement of an arm that is adjacent the shoulder support member.

26. The method of claim 25, wherein the over the shoulder strap is attached to an upper portion of the shoulder support member and the under the shoulder strap and the arm strap are attached to a portion of the shoulder support member, under the over the shoulder strap.

27. The method of claim 25, further comprising providing a motion-limiting restraint strap and attaching the motion-limiting restraint strap to the arm band or to the sleeve and a front segment of the torso strap or the torso portion of the brace body.

28. A method of providing a customizable shoulder brace for treating a subject at risk for shoulder dislocation or needing support of a shoulder, comprising:
providing a shoulder brace;
shaping a shoulder support member to conform to a shape of external anatomy over a glenohumeral joint of a shoulder of a patient; and
placing the shaped thermoplastic shoulder support member anterior or posterior to a user's shoulder over the glenohumeral joint and holding the shoulder support member in position using the shoulder brace.

29. The method of claim 28, wherein the providing step comprises providing at least one moldable shoulder support member in a kit to allow for custom molding of a respective shoulder support member for a respective user, wherein the thermoplastic shoulder support member has a thickness between about 0.125 inches to about 0.25 inches.

30. The method of claim 29, wherein the at least one moldable shoulder support member is a thermoplastic shoulder support member that is malleable for between 3-5 minutes when heated to between about 65 degrees Celsius to about 70 degrees Celsius for about 1 minute.

31. The method of claim 28, wherein the at least one moldable shoulder support member is provided in the kit pre-attached to three strap segments including an over the shoulder strap segment, an under the shoulder strap segment and an arm strap segment.

32. The method of claim 31, wherein the under the shoulder back strap segment and the arm strap segment are fixedly attached to a lower end portion of the shoulder support member and the over the shoulder back strap segment is fixedly attached to an upper end portion of the shoulder support member.

33. The method of claim 28, wherein the shoulder brace comprises a brace body having a shirt configuration with at least one sleeve and at least one pocket for releasably engaging the shoulder support member.

34. The method of claim 28, wherein the shoulder brace comprises only a plurality of cooperating straps.

35. The method of claim 28, wherein the shoulder support member comprises a plurality of laterally extending slots that engage straps of the shoulder brace.

36. The method of claim 31, wherein the over the shoulder strap segment and the under the shoulder strap segment have a stretch in a range of 40% to 60%.
